(19) United States
(12) Patent Application Publication
(10) Pub. No.: US 2015/0044330 A1
(43) Pub. Date: Feb. 12, 2015

(54) Strains of Lactobacillus Rhamnosus GG Capable of Growth on Lactose and the Uses Thereof

(71) Applicant: GLYCOSYN LLC, Waltham, MA (US)

(72) Inventors: Matthew Ian Heidtman, Brighton, MA (US); Massimo Merighi, Somerville, MA (US); John M. McCoy, Reading, MA (US)

(21) Appl. No.: 14/453,153

(22) Filed: Aug. 6, 2014

Related U.S. Application Data
(60) Provisional application No. 61/862,603, filed on Aug. 6, 2013.

(51) Int. Cl. A23C 9/123; A23L 1/30; C12N 15/74

(52) U.S. Cl. CPC A23C 9/123; C12N 15/746; A23L 1/3014; A23Y 2220/73
USPC 426/43; 435/252.3; 435/34; 426/61

(57) ABSTRACT

The invention provides compositions and methods utilizing an improved strain of bacteria, a Lac+ Lactobacillus rhamnosus strain of bacteria.

### RELATED APPLICATIONS

This application claims priority to U.S. Provisional Application Ser. No. 61/862,603 filed Aug. 6, 2013. The disclosure of which is hereby incorporated by reference in its entirety herein.

### FIELD OF THE INVENTION

The field of the invention relates to probiotic microorganisms.

### INCORPORATION-BY-REFERENCE OF SEQUENCE LISTING

The contents of the text file named “37847513001US_ST25.txt”, which was created on Aug. 4, 2014 and is 36.0 KB in size, are hereby incorporated by reference in their entireties.

### BACKGROUND OF THE INVENTION

Probiotics are generally defined as live microorganisms that confer a health benefit to the host when administered in adequate amounts (Sanders, 2003, Nutr Rev 61, 91-99; Sullivan and Nord, 2005, J Intern Med 257, 78-92). Evidence exists suggesting that probiotic bacteria and yeast strains can be beneficial in treating a wide range of adverse health conditions, such as diarrhea, food allergies, dental caries and respiratory infections (Goldin and Gorbach, 2008, Clin Infect Dis 46 Suppl 2, S96-100; discussion S144-51). The effects of probiotics can be mediated through direct or indirect, mechanisms; for example directly through influencing gut flora composition or indirectly through subsequent provision of metabolites (nutrients, vitamins) to the host by the gut flora, or through modulation of host immune system functions (Sanders, 2000, J Nutr 130, 384S-390S; Sanders, 2003, Nutr Rev 61, 91-99).

### SUMMARY OF THE INVENTION

The invention features an improved strain of bacteria, a Lac+ Lactobacillus rhamnosus bacterial strain, e.g., one comprising at least 95%, 96%, 97%, 98%, 99%, 99.1%, 99.2%, 99.3%, 99.4%, 99.5%, 99.6%, 99.7%, 99.8%, 99.9% of the nucleotide sequence of ATCC 53103 and further comprising a gene encoding a functional LacG protein and a gene encoding a functional LacT protein or a lacTEGF operon lacking a functional transcriptional terminator within 100 nucleotide base pairs from the initiating codon of lacT. For example, the bacterial strain comprises at least 99% of the nucleotide sequence of ATCC 53103.

Also within the invention is a Lactobacillus rhamnosus bacterial strain comprising at least 99% of the nucleotide sequence of strain ATCC 53103 and further comprising one or more of the following: at least one mutation that suppresses the LacT defect found in the strain ATCC 53103; at least one mutation in the transcriptional terminator regulating the lacTEGF operon, wherein the at least one mutation inactivates the transcriptional terminator; the nucleotide sequence encoding a functional LacT; and the nucleotide sequence encoding a functional LacG. For example, the bacterial strain comprises at least 99.9% of the nucleotide sequence of ATCC 53103.

In one aspect, the gene encoding a functional LacG protein comprises the amino acid sequence SEQ ID NO: 6 or 8. In another aspect, the gene encoding a functional LacG protein comprises the nucleotide sequence SEQ ID NO: 5 or 7. In a further aspect, the gene encoding a functional LacT protein comprises the amino acid sequence SEQ ID NO: 11. In a further aspect, the gene encoding a functional LacT protein comprises the nucleotide sequence SEQ ID NO: 10. In another aspect, the transcriptional terminator comprises any one of the nucleotide sequences SEQ ID NO: 12-15. The strains described herein are non-naturally occurring.

An ingestible composition comprising lactose and the above-described strain is also encompassed by the invention. For example, the compositions are formulated as infant formula, milk drink, whey protein drink, sports drink, buttermilk, cheese, yogurt, drinkable yogurt, baby food, weaning food, or a confection such as candy or gum, as well as a yogurt starter culture. Given the ability to grow on and metabolize lactose, the strain is also used in a method of reducing the symptoms of lactose intolerance. According to the method, the strain, e.g., alone or in an ingestible product containing lactose is administered to an individual that comprises or has lactose intolerance or has been diagnosed with the disorder. An individual that has lactose intolerance is not limited by diagnosis; those that have lactose intolerance may experience or suffer from varying severity of symptoms that arise after ingesting dairy products that contain lactose. Such symptoms include, but are not limited to abdominal bloating and cramps, flatulence, diarrhea, nausea, borborygmi (rumbling stomach), and vomiting. These symptoms may arise thirty minutes to two hours, or more, after consumption. The severity of symptoms typically increases with the amount of lactose consumed.

The present invention further features a method for identifying a bacteria that digests lactose by transforming a Lactobacillus rhamnosus with a first plasmid comprising functional or wild-type LacT and a selective marker; selecting for at least one Lac+ colony by growing the transformants of the first step on plates containing lactose and the selective marker, passaging the Lac+ colony of the previous step at least two times in the absence of lactose and the selective marker, such that the Lac+ colonies no longer contain the first plasmid; growing the Lac+ colony of the previous step on plates containing lactose.

As used herein, the term “LGG” refers to Lactobacillus rhamnosus strain GG (ATCC 53103). The mutant LGG bacteria described herein is a non-natural variant of LGG.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Percent identity is determined using search algorithms such as BLAST and PSI-BLAST (Altschul et al., 1990, J Mol Biol 215:3, 403-410; Altschul et al., 1997, Nucleic Acids Res 25:17, 3389-402). For the PSI-BLAST search, the following exemplary parameters are employed: (1) Expect threshold was 10; (2) Gap cost was Existence:11 and Extension:1; (3) The Matrix employed was BLOSUM62; (4) The filter for low complexity regions was “on”.

The invention also features a vector, e.g., a vector containing a nucleic acid. The vector can further include one or more regulatory elements, e.g., a heterologous promoter. The regulatory elements can be operably linked to a gene encoding a protein, a gene construct encoding a fusion protein gene, or a series of genes linked in an operon in order to express the fusion protein. In yet another aspect, the invention comprises an isolated recombinant cell, e.g., a bacterial cell containing an aforementioned nucleic acid molecule or vector. The nucleic acid is optionally integrated into the genome.

Polynucleotides, polypeptides, and oligosaccharides of the invention are purified and/or isolated. Purified defines a degree of sterility that is safe for administration to a human subject, e.g., lacking infectious or toxic agents. Specifically, as used herein, an “isolated” or “purified” nucleic acid molecule, polynucleotide, polypeptide, protein or oligosaccharide, is substantially free of other cellular material, or culture medium when produced by recombinant techniques, or chemical precursors or other chemicals when chemically synthesized. For example, purified compositions are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, thin layer chromatography, or high-performance liquid chromatography (HPLC) analysis. For example, a “purified protein” refers to a protein that has been separated from other proteins, lipids, and nucleic acids with which it is naturally associated. Preferably, the protein constitutes at least 10, 20, 50, 70, 80, 90, 95, 99-100% by dry weight of the purified preparation.

Similarly, by “substantially pure” is meant an oligosaccharide that has been separated from the components that naturally accompany it. Typically, the oligosaccharide is substantially pure when it is at least 60%, 70%, 80%, 90%, 95%, or even 99%, by weight, free from the proteins and naturally-occurring organic molecules with which it is naturally associated.

By “isolated nucleic acid” is meant a nucleic acid that is free of the genes which, in the naturally-occurring genome of the organism from which the DNA of the invention is derived, flank the gene. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones.

A “heterologous promoter” is a promoter which is different from the promoter to which a gene or nucleic acid sequence is operably linked in nature.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

By the terms “effective amount” and “therapeutically effective amount” of a formulation or formulation component is meant a nontoxic but sufficient amount of the formulation or component to provide the desired effect.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a diagram showing regulation and function of the lacTEGF operon in L. rhamnosus strain GG (ATCC 53103) (LGG). LacT and LacF together function as a phosphoenolpyruvate-dependent phosphotransferase system. LacG is a phospho-β-galactosidase and LacT is a transcriptional antiterminator. In the absence of lactose, LacEF, acting together, phosphorylate LacT, inactivating the protein which results in an inhibition of transcription through the operon due to the transcriptional terminator (represented by t1 in the figure). In the presence of lactose, the LacEF complex preferentially phosphorylates lactose, allowing LacT to escape phosphorylation. Non-phosphorylated LacT is the active form of the protein and can bind to the RAT site, preventing terminator formation and allowing complete full-length transcription of the operon to occur (Alpert and Siebers, 1997, J Bacteriol 179, 1555-562; Gosalbes et al., 1999, J Bacteriol 181, 3928-934; Tsai and Lin, 2006, J Appl Microbiol 100, 446-459; Tsai et al., 2009, Microbiology 155, 751-760).

FIG. 2 is a diagram showing inactivating mutations in the lacTEGF operon of L. rhamnosus strain GG (ATCC 53103). Comparison of the LGG genomic sequence to the genomic sequence of strain L. rhamnosus Lc705 revealed a 4 base-pair deletion in lacT immediately following nucleotide 96 (Kankainen et al., 2009, Proc Natl Acad Sci USA 106, 17193-98). This deletion results in a frameshift mutation that leads to the introduction of several erroneous amino acids and then a premature stop codon after amino acid 37. An additional mutation in the operon occurs in lacG and is a cytosine to thymine change at nucleotide 691. This mutation introduces a premature stop codon and truncates the protein prematurely after amino acid 230 (Kankainen et al., 2009, Proc Natl Acad Sci USA 106, 17193-98). The nucleotide sequences of mutant lacT and lacG and the amino acid sequences of mutant LacT and LacG are shown in Table 2.

FIG. 3 is a pair of photographs showing the isolation of Lac+ Lactobacillus strain GG (ATCC 53103) revertants. LGG was transformed with pG362 (pTRKH3-ermlacT) to generate strain L9. Strain L9 was plated to MRS media containing 2% lactose and 5 μg/ml erythromycin (5×10⁷ cells to each of 12 plates) and incubated for 4 days at 37° C. in an atmosphere containing 2% CO₂. At the end of the incubation period, 10 Lac+ LGG colonies were obtained (representative plates shown above, arrows indicate positions of Lac+ LGG colonies). Each Lac+ isolate was re-streaked onto fresh MRS plates containing 2% lactose and 5 μg/ml erythromycin and each was found to be reproducibly Lac+.

FIG. 4A is a bar graph showing liquid growth analysis of Lac+ LGG strains isolated from plating of strain L9 (LGG transformed with pG362). Lac+ isolates (designated T1 through T10) were assessed for growth in MRS media containing 2% dextrose with 5 μg/ml erythromycin or MRS media containing 2% lactose with 5 μg/ml erythromycin. Strains were grown for 48 h at 37° C. in an atmosphere containing 2% CO₂ and the OD₆₀₀ of each culture then measured with a spectrophotometer. LGG transformed with an empty vector was tested in parallel as a control (strain L8). As shown above, the negative control strain grew in the presence of dextrose but not lactose (Lac− phenotype), while 8 of 10 Lac+ isolates grew robustly when lactose was provided as the sole sugar source (Lac+ phenotype).

FIG. 4B is a table showing Lac+ isolates T7 (later designated L11) and T8 (later designated L12) were selected for further analysis. Genomic DNA was isolated from these strains, the lacG coding region amplified by PCR and subjected to DNA sequencing. Point mutations within the premature stop codon within lacG were identified and are summarized in the table. The sequences of the Lac+ isolates are shown in Table 2.

FIG. 5 is a pair of photographs showing isolation of plasmid-independent Lac+ LGG strains. Strains L13 (lacG with premature stop codon at position 231 reverted to lysine) and L14 (lacG with premature stop codon at position 231 reverted to glutamine) were plated to MRS media containing 2% lactose as the sole sugar source (1×10⁸ cells plated for each strain). Plates were incubated at 37° C. for 4 days in an atmosphere containing 2% CO₂. Approximately 1000 Lac+ colonies were isolated from the plating of each strain. Five Lac+ colonies from derived from either the plating of L13 or L14 were picked and re-streaked to MRS plates containing 2% lactose. All candidates grew robustly upon re-streaking, confirming their Lac+ phenotype.

FIG. 6 is a bar graph showing growth analysis of plasmid-independent Lac+ isolates in liquid culture. Five Lac+ isolates derived from parental strains L13 and L14 were tested for growth in liquid MRS medium containing either 2% glucose or 2% lactose as the sole sugar source. Cultures were incubated for 48 h at 37° C. in an atmosphere containing 2% CO₂. The control LGG parental strain (strain L3) was unable to utilize lactose for growth, while 9 of 10 Lac+ isolates derived from L13 and L14 grew well in liquid media with lactose as the sole sugar source. Isolates L13-1 and L13-2 (later designated as L15 and L16), as well as L14-4 and L14-5 (later designated as L17 and L18) were selected for further characterization by DNA sequencing of the lacT region.

FIG. 7A is a diagram showing nucleotide sequence of the Rho-independent transcriptional terminator upstream of the lacTEGF operon. The sequence of the adjacent RAT (ribonucleic antiterminator) site is also shown. The position of the guanosine nucleotide at the 3′ end of the terminator sequence mutated in Lac+ LGG strains L15, L16, L17 and L18 is denoted by the black arrow.

FIG. 7B is a table summarizing the specific mutations in the transcriptional terminator identified by DNA sequencing in Lac+ LGG strains L15, L16, L17 and L18.

FIG. 7C is a diagram showing a stem-loop structure of the transcriptional terminator (Tsai et al., 2009, Microbiology 155, 751-760) and position of the guanine nucleotide mutated in the Lac+ LGG isolates. The sequences of the terminator for strains L15, L16, L17 and L18 are show in Table 2.

FIG. 8 is a diagram of plasmid pG362 map. The pG362 nucleotide sequence is found in Table 2 (SEQ ID NO: 9). The nucleic acid sequence of lacT from the Lc705 strain (SEQ ID NO: 10) and the amino acid sequence of LacT from the Lc705 strain (SEQ ID NO: 11) can be found in Table 2.

### DETAILED DESCRIPTION

Bacteria of the genus Lactobacillus, which are a subset of the lactic acid producing bacteria, are commonly used in the production of a wide variety of fermented milk products, such as soured milk, cheese and yogurt. The Lactobacilli are also frequently utilized as probiotics (Reid, 1999, Appl Environ Microbiol 65, 3763-66). In particular, the strain Lactobacillus rhamnosus strain GG (ATCC 53103, referred to herein as LGG), first isolated in 1985 from human intestinal flora (Goldin and Gorbach, 2008, Clin Infect Dis 46 Suppl 2, S96-100; discussion S144-51; U.S. Pat. No. 4,839,281; hereby incorporated by reference), is one of the most well studied probiotic strains to date. LGG is particularly well suited for probiotic use in humans due to its remarkable ability to survive exposure to stomach acid and to bile, to transit with good viability to the small and large intestine, and there to adhere efficiently to human gut epithelia (Alander et al., 1999, Appl Environ Microbiol 65, 351-54; Goldin and Gorbach, 2008, Clin Infect Dis 46 Suppl 2, S96-100; discussion S144-51; Kankainen et al., 2009, Proc Natl Acad Sci USA 106, 17193-98). Strong evidence now exists to suggest that LGG can provide benefits in the treatment of a variety of medical conditions, including; infectious diarrhea in children and adults, antibiotic associated diarrhea, traveler's diarrhea, respiratory infections, and enhancement of immune response following vaccination (Hatakka et al., 2001, BMJ 322, 1327; Alvarez-Olmos and Oberhelman, 2001, Clin Infect Dis 32, 1567-576; Sanders, 2003, Nutr Rev 61, 91-99; Vendt et al., 2006, J Hum Nutr Diet 19, 51-58). These studies have provided impetus for inclusion of LGG into a wide variety of commercially available probiotic products. Such products include orally administered probiotic capsules containing lyophilized LGG cells, infant formula, fermented milk and whey drinks, fruit juices, sports drinks, yogurts, buttermilk, and semi-hard cheeses.

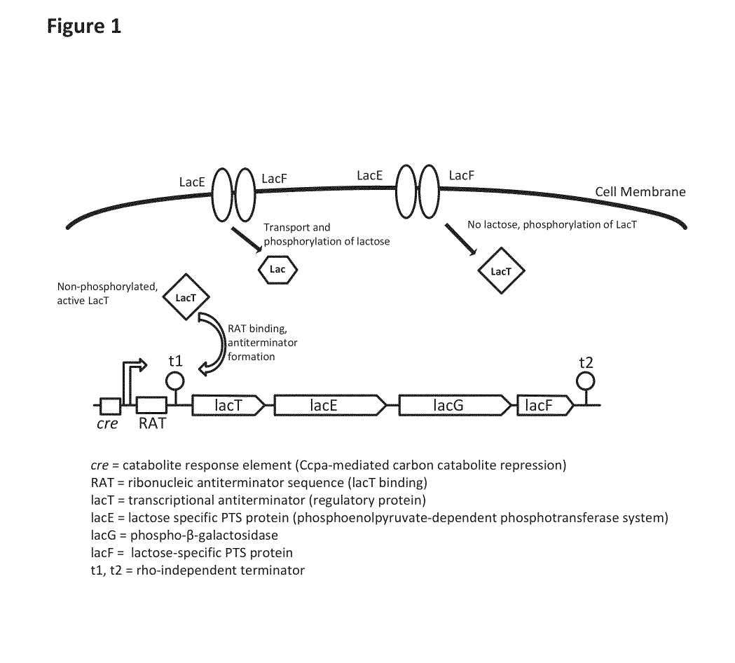

Despite LGG's definitive classification as a lactic acid bacterium, the vast majority of which are known to catabolize lactose for growth, LGG itself is unable to metabolize lactose (a phenotype termed “Lac−”) (Kankainen et al., 2009, Proc Natl Acad Sci USA 106, 17193-98; Pessione, 2012, Front Cell Infect Microbiol 2). The genome of LGG (ATCC 53103) was compared to the closely related genome of L. rhamnosus strain Lc705 (Kankainen et al., 2009, Proc Natl Acad Sci USA 106, 17193-98, and available from the Valio culture collection, Valio Ltd.). Lc705, in contrast to LGG, is known to utilize lactose for growth (a phenotype termed “Lac+”) and is used routinely as an adjunct starter culture in dairy products (Hatakka et al., 2008, J Am Coll Nutr 27, 441-47). This comparison revealed that LGG possesses two distinct mutations in its chromosomal lactose utilization operon. This operon is made up of 4 genes termed lacT, lacE, lacG, and lacF (lacTEGF). The lacE and lacF gene products together form the lactose-specific phosphoenolpyruvate-dependent phosphotransferase system (Lac-PTS) responsible for lactose uptake (Breidt et al., 1987, J Biol Chem 262, 16444-49; de Vos et al., 1990, J Biol Chem 265, 22554-560). When transported into the cell via the Lac-PTS, lactose is phosphorylated and then hydrolyzed by LacG (a phospho-β-galactosidase) to produce glucose and galactose-6-phosphate (De Vos and Gasson, 1989, J Gen Microbiol 135, 1833-846; Honeyman and Curtiss, 1993, J Gen Microbiol 139, 2685-694). Glucose, after initial phosphorylation, can then be catabolized by the Embden-Meyerhof glycolytic pathway to fuel cellular metabolism, while galactose-6-phosphate can be catabolized via the tagatose-6-phosphate pathway (Tsai and Lin, 2006, J Appl Microbiol 100, 446-459). LacT functions as a transcriptional antiterminator that allows complete transcription of the lacTEGF operon to occur in a lactose-dependent manner (Gosalbes et al., 2002, Microbiology 148, 695-702). Specifically, in the presence of lactose, LacT is no longer subject to phosphorylation by the Lac-PTS and is able to bind to a RAT (ribonucleic antiterminator) site present in the lacTEGF operon promoter region. Binding of LacT to this RAT site prevents formation of a downstream stem-loop structure which otherwise would cause premature transcription termination and abolish expression of the downstream lacTEGF gene products. Thus the presence of lacT allows for efficient and complete transcription though the operon and expression of the lactose utilization genes. This mode of action is likely to be similar to antiterminator proteins from other bacteria, such as BglG of E. coli, and SacT, SacY, LicT and GlcT of B. subtilis (Alpert and Siebers, 1997, J Bacteriol 179, 1555-562; Gosalbes et al., 1999, J Bacteriol 181, 3928-934; Gosalbes et al., 2002, Microbiology 148, 695-702; Santangelo and Artsimovitch, 2011, Nat Rev Microbiol 9, 319-329). Regulation of the lacTEGF operon by LacT is depicted in FIG. 1.

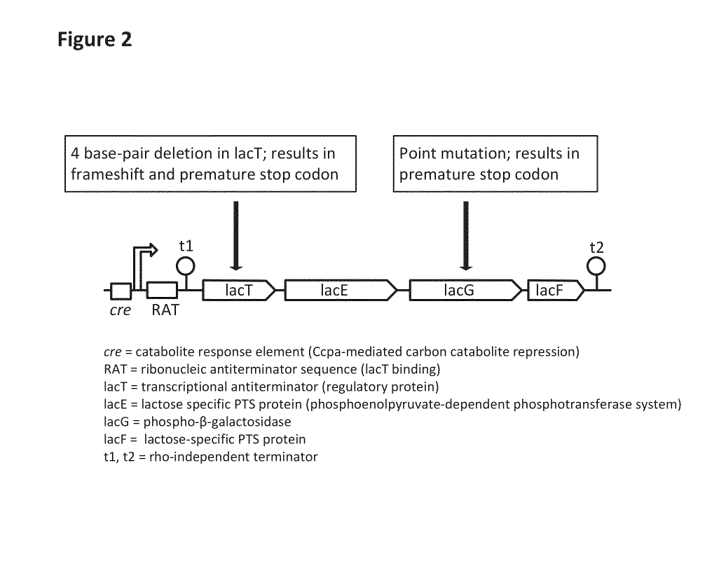

LGG is Lac− by virtue of 2 separate mutations in the lacTEGF operon. The first mutation is in lacT and is a four base pair deletion immediately following nucleotide 96 (SEQ ID NO: 1). This deletion results in a frameshift mutation that leads to the introduction of several erroneous amino acids and finally a premature translational stop codon after amino acid 37 (SEQ ID NO: 2). The end result of the deletion is expression of a truncated, non-functional protein that cannot promote antitermination at the lacTEGF promoter region RAT site (Kankainen et al., 2009, Proc Natl Acad Sci USA 106, 17193-98). The second mutation lies in lacG and is a cytosine to thymine point mutation at nucleotide 691 of the gene sequence. This mutation changes a CAG codon (glutamine) to TAG (translation stop) and truncates the protein prematurely after amino acid 230. Thus, LGG lacks both a functional phospho-β-galactosidase (LacG) to catabolize phosphorylated lactose, and a functional LacT to promote transcription through the lacTEGF operon. The approximate positions of each mutation within the lacTEGF operon are depicted in FIG. 2. The nucleotide sequences of LacT (SEQ ID NO: 1) and LacG (SEQ ID NO: 3) and the amino acid sequences of mutated LacT (SEQ ID NO: 2) and LacG (SEQ ID NO: 4) are shown in Table 2.

### Improved Lactobacillus Strain GG (ATCC 53103)

Given that LGG is unable to digest lactose for growth, and yet is provided in several products where lactose is abundant (i.e. infant formula, milk/whey drinks, cheeses etc.), a Lac+ variant of LGG was developed. This improved strain offers considerable advantages over the parental Lac− strain. Such a Lac+ LGG strain possesses all of the advantages of the parental strain (i.e. ability to adhere to human intestinal epithelia, ability to survive stomach acid/intestinal bile, and passage intact to the intestines), yet is able to use lactose for growth and therefore colonizes the host to a greater extent. This feature of more robust growth and subsequent more efficient colonization of the host provides an enhanced probiotic benefit, due to higher numbers of surviving and persisting LGG bacteria in the host. These characteristics are particularly useful in a situation where LGG is provided in the context of a product where lactose is readily available (i.e. infant formula, milk/whey drinks, buttermilk, cheese, and yogurt).

Therefore, the uses of a Lac+ LGG strain are as follows: (1) Such a strain is useful as a probiotic in a variety of lactose-containing dairy products such as infant formula, powdered milk, milk and whey drinks, buttermilk, cheese and yogurts. In this context, a Lac+ LGG strain offers a significant advantage over the parental strain, as such a strain is able to metabolize the lactose present in these products when they are administered together and therefore grows to higher density within the host. (2) A Lac+ version of LGG is useful as a starter culture in the dairy industry for production of the products mentioned previously, including but not limited to fermented milk and whey drinks, cheeses, buttermilks and yogurts. This is a significant advantage over the parental LGG strain, as this strain is Lac− and therefore would not be useful for a starter culture. (3) A Lac+ version of LGG is useful for treating lactose intolerance when provided as an additive (or starter culture) to yogurt, milk/whey drinks, or other lactose containing dairy products. Lactose intolerance is a common condition caused by malabsorption of lactose in the colon due to an intestinal lactase deficiency (Montalto et al., 2006, World Journal of Gastroenterology 12, 187). When lactose is not digested in the small intestine, it passes through the gastrointestinal tract to the colon where it can lead to symptoms of lactose intolerance. These symptoms can include abdominal pain, bloating, flatulence, nausea, and diarrhea. These symptoms result from colonic fermentation of unabsorbed lactose (Lomer et al., 2008, Aliment Pharmacol Ther 27, 93-103). Unabsorbed lactose in the colon can also cause an increased osmotic load and lead to a greater secretion of fluid into the colon, resulting in loose stools and diarrhea (Lomer et al., 2008, Aliment Pharmacol Ther 27, 93-103; Jellema et al., 2010, QJM 103, 555-572). Evidence exists indicating that an appropriate strain of lactic acid bacterium administered in adequate amounts can alleviate symptoms of lactose intolerance (He et al., 2008, J Appl Microbiol 104, 595-604). Specifically, Lac+ lactic acid bacteria digest some of the lactose present in dairy products, and therefore ameliorate the discomfort caused by lactose malabsorption. In addition Lac+ LGG given its ability to survive transit through the gastro-intestinal (GI) tract resides in the small intestine and colon in significant numbers. As a consequence of this survival and adherence to the intestinal epithelium, Lac+ LGG ferments non-absorbed lactose “in situ” and thus further ameliorate the symptoms associated with lactose intolerance. Thus, a lactose-containing dairy product containing Lac+ LGG is better tolerated in the gastrointestinal tract in a lactose-intolerant individual as compared to a dairy product lacking this strain. A Lac+ LGG strain therefore serves as an effective means of alleviating the symptoms of lactose intolerance while at the same time providing the additional well-characterized probiotic effects of LGG.

The present invention also features mixtures comprising the Lac+ LGG strain disclosed herein and other commercial yogurt strains, e.g., Acetobacter orientalis, Bifidobacterium animalis, Bifidobacterium lactis, Bifidobacterium bifidum, Lactobacillus acidophilus, Lactobacillus bulgaricus, Lactobacillus casei, Lactobacillus delbrueckii subsp. Bulgaricus, Lactobacillus delbrueckii subsp. Lactis, Lactobacillus lactis subsp. Cremoris, Lactobacillus rhamnosus, Lactococcus lactis, Leuconostoc mesenteroides, Streptococcus lactis var. bollandicus, Streptococcus taette, and Streptococcus thermophiles.

### Construction of Lac+ Lactobacillus Strain GG (ATCC 53103)

As an initial approach to construct a Lac+ LGG strain, large numbers (2×10¹⁰ colony forming units (c.f.u).) of LGG (strain L3) cells were plated onto MRS media containing 2% lactose as the sole sugar source and incubated the plates for 4 days at 37° C. in an atmosphere containing 2% CO₂. With this approach, the inherent error rate of DNA polymerase during chromosomal replication would lead to revertants in lacG (Kunkel, 2009, Cold Spring Harbor Symposia on Quantitative Biology 74, 91-101). The mutation in lacT is a four base-pair deletion, which is essentially non-revertable (Drake, 1991, Proc Natl Acad Sci USA 88, 7160-64; Kunkel, 2004, J Biol Chem 279, 16895-98). However, we hypothesized that additional base pair deletions might occur within lacT that might restore the lacT reading frame in a functional manner. Regardless, we were unable to recover any Lac+ LGG colonies using this approach. This is likely because the probability of recovering revertants in both lacG and lacT simultaneously within the same cell is extremely low.

As an alternative approach to isolate revertant Lac+ LGG strains, a plasmid was constructed in which a wild-type version of lacT is expressed from the constitutive erm (enterococcal rRNA adenine N-6-methlytransferase) promoter and plasmid selection is maintained by providing 5 μg/ml erythromycin in the culture medium. A map of this plasmid (pG362) and its nucleotide sequence is shown in Table 2.

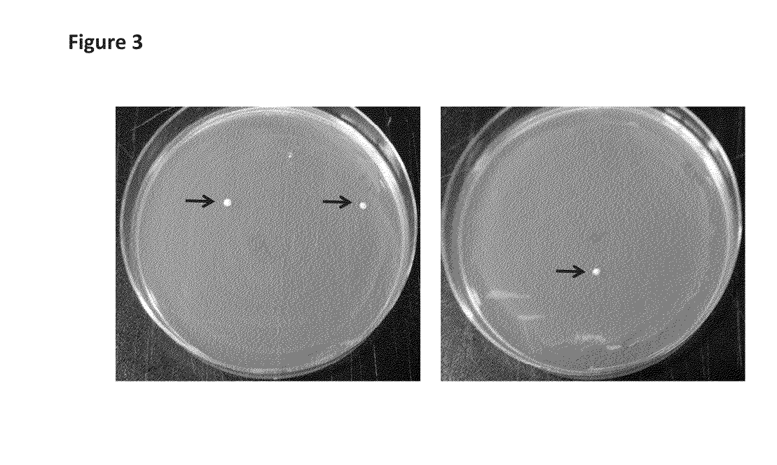

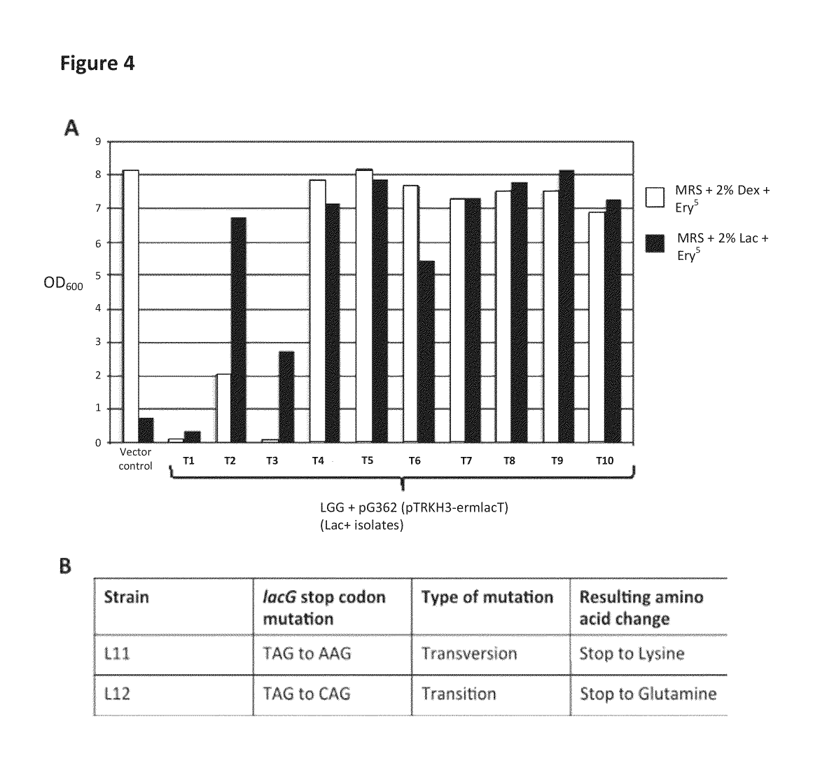

LGG (ATCC 53103) was transformed with plasmid pG362 to generate strain L9. With this approach, complementing the chromosomal lacT mutation in LGG with a plasmid-borne wild-type version of lacT permitted ease of isolation of revertants in lacG, which were observed as Lac+ colonies on lactose-containing plates. To this end, a total of 6×10⁸ cells of strain L9 were plated to MRS plates containing 2% lactose and 5 μg/ml erythromycin to maintain plasmid selection. After incubation for 4 days at 37° C. in an atmosphere containing 2% CO₂, the growth of 10 Lac+ colonies (see FIG. 3 for a representative plate image) was observed. These colonies grew robustly upon re-streaking onto MRS/lactose/erythromycin plates, confirming the Lac+ phenotype. Morphologically, when inspected microscopically, isolated cells from these Lac+ colonies were indistinguishable from the parental LGG strain. In order to obtain a more quantitative measure of growth, the growth of these Lac+ LGG isolates in liquid MRS media containing either dextrose (glucose) or lactose as a sugar source was observed. As shown in FIG. 4A, the parental LGG strain transformed with an empty vector (strain L8) failed to grow in media containing lactose, while 8 of the 10 Lac+ isolates from the above selection strategy grew in media containing lactose as the sole sugar source. Therefore, by complementing the chromosomal mutation in lacT with a plasmid-borne wild-type copy, Lac+ LGG revertants were isolated using a plating strategy onto media containing lactose as the sole sugar source.

The Lac+ isolates obtained from this approach were likely to be revertants in lacG, specifically they would be single nucleotide changes (caused by rare but expected DNA polymerase errors arising during normal DNA replication) and lying within the LGG lacG premature stop codon. These single nucleotide changes result in a transformation of the LGG lacG stop codon to a codon encoding a functional amino acid. In order to test this idea, genomic DNA was isolated from Lac+ isolates T7 and T8 (later designated strains L11 and L12 respectively). The lacG region of these strains was amplified by PCR and subjected to DNA sequencing. The analysis revealed a single point mutation in this region for each strain that reverted the premature stop codon in lacG to a functional codon. Specifically, for strain L11, the thymine at position 691 in lacG mutated to an adenine. This resulted in a change of the codon at this position from TAG (stop) to AAG (lysine). The nucleotide sequence of this version of lacG (SEQ ID NO: 5) and the amino acid sequence (SEQ ID NO: 6) are provided in Table 2. For strain L12, the thymine at position 691 in lacG mutated to a cytosine. This results in a change of the codon at this position from TAG (stop) to CAG (glutamine). The nucleotide sequence of this version of lacG (SEQ ID NO: 7) and the amino acid sequence (SEQ ID NO: 8) are provided in Table 2. In summary, two of the Lac+ LGG isolates obtained by plating strain L9 to media containing lactose were found to have mutations in the premature stop codon within lacG, reverting this codon to one that encodes a functional amino acid (FIG. 4B). Thus, the combination of a functional version of LacG with wild-type LacT provided in trans by plasmid pG362 was sufficient to generate a Lac+ phenotype. The nucleotide sequence of wild-type LacT from L. rhamnosus strain Lc705 is provided in SEQ ID NO: 10 and the amino acid sequence of wild-type LacT from L. rhamnosus strain Lc705 is provided in SEQ ID NO: 11.

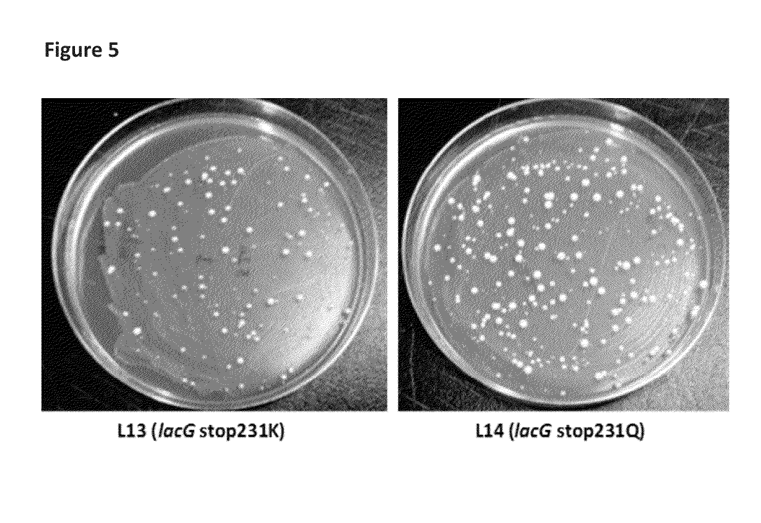

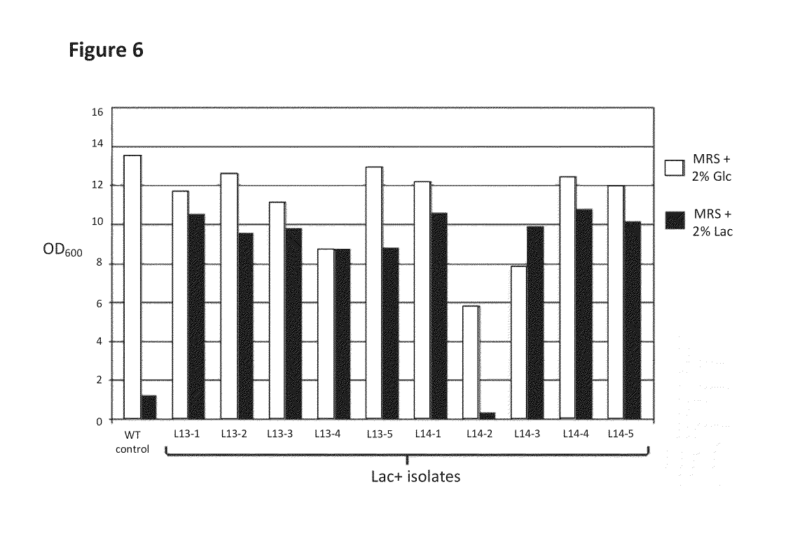

Since the approach permitted the generation of a functional allele of lacG in LGG, we hypothesized that a second round of plating and selection in this new strain background might allow us to select for additional mutations that would generate Lac+ LGG isolates in a plasmid-independent manner. To this end, we first passaged strains L11 and L12 three times in the absence of selective pressure (without erythromycin or lactose) to enable these cells to lose plasmid pG362. The resulting strains (L13 and L14) were then assessed for the presence of plasmid pG362 by testing for loss of erythromycin resistance as well as by PCR with plasmid-specific primers. These tests indicated that both strains had successfully lost plasmid pG362. Strains L13 and L14 were also observed to be Lac−, further evidence that these strains had lost plasmid pG362. Next, 1×10⁸ cells of L13 and L14 were plated to MRS media containing 2% lactose and incubated for 4 days at 37° C. in an atmosphere containing 2% CO₂. After the incubation period, the growth of approximately 1000 Lac+ colonies from the plating of strain L13 and 1000 Lac+ colonies from the plating of strain L14 (See FIG. 5 for a representative plate) was observed. Five Lac+ colonies derived from each parental strain were picked and re-streaked to MRS containing 2% lactose. These colonies grew robustly upon re-streaking, confirming their Lac+ phenotype. In order to obtain a more quantitative measure of growth, the growth of these, second round, Lac+ LGG isolates in liquid MRS media containing either dextrose or lactose as a sugar source was examined. As shown in FIG. 6, the parental LGG strain failed to grow in media containing lactose, while 9 of the 10 Lac+ isolates from this second selection grew robustly in media containing lactose as the sole sugar source. The strains described herein comprise mutations that were induced by selective pressures exerted by specifically artificially designed and constructed growth conditions that do not occur in nature, e.g., conditions not encountered in the colon or feces of a mammalian, e.g., human subject.

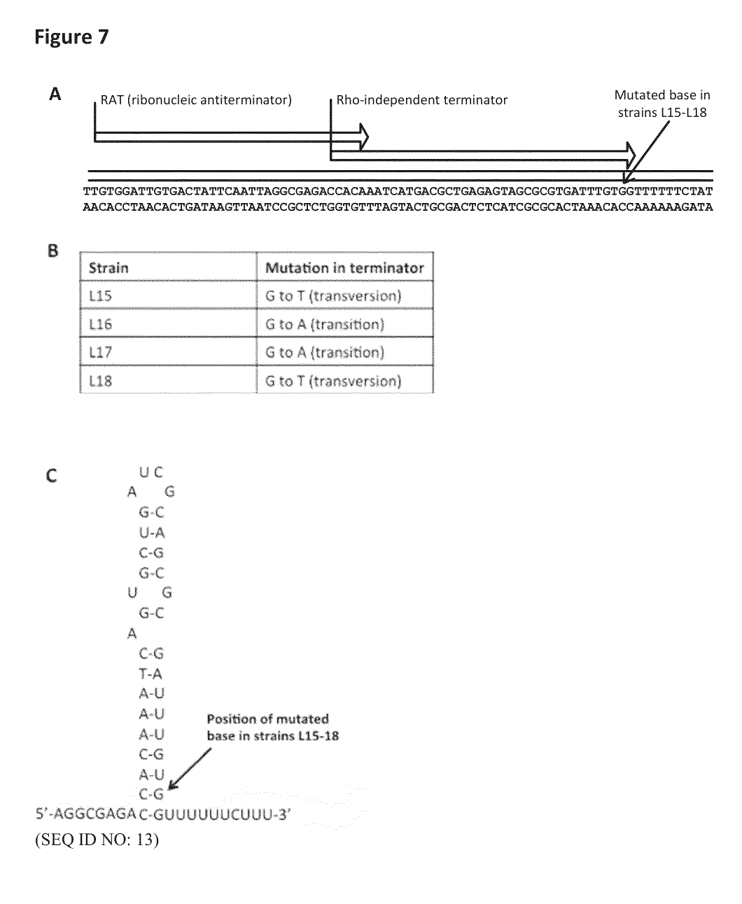

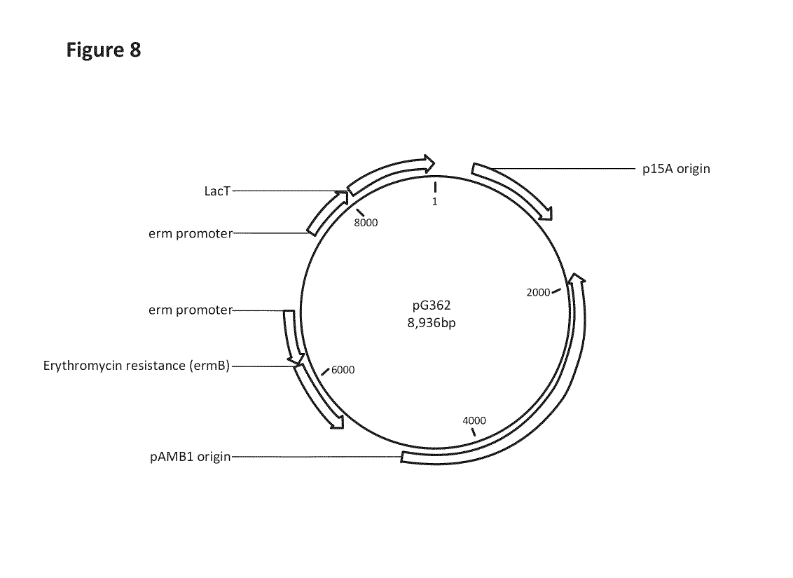

Studies were then undertaken to elucidate the genetic changes that occurred in this second group of Lac+ isolates. Given the large number of Lac+ colonies obtained from plating both L13 and L14, inactivating mutations may have arisen in the transcriptional terminator regulating the lacTEGF operon. This region is predicted to contain 39 base pairs that form a stem-loop structure that inhibits transcription (Tsai et al., 2009, Microbiology 155, 751-760). Changes in any one of these 39 bases might disrupt formation of the stem-loop structure and allow unimpeded transcription of the operon. Thus, this transcriptional terminator region is a large target for potential revertant single nucleotide changes to arise, which could explain relatively large number of Lac+ LGG isolates obtained in this second round of plating. To test this possibility, 2 Lac+ isolates each was selected from the plating of L13 (designated L15 and L16) and L14 (designated L17 and L18). Genomic DNA was prepared from these strains and amplified lacT and its upstream promoter region by PCR. These PCR products were then subjected to DNA sequencing to identify any mutations that may have arisen. Indeed, a single point mutation was observed in the predicted transcriptional terminator region for each strain. These mutations for the four revertant isolates tested were all at the same position within the transcriptional terminator, arising at the 3′ end of the sequence where the base of the stem-loop structure is predicted to form (FIG. 7) (Tsai et al., 2009, Microbiology 155, 751-760). The sequences of the mutated transcriptional terminator sequence for L15 (SEQ ID NO: 12); L16 (SEQ ID NO: 13); L17 (SEQ ID NO: 14); and L18 (SEQ ID NO: 15) are provided in Table 2. These mutations prevent the efficient formation of the stem-loop structure, and therefore allow transcription through the lacTEGF operon in the absence of functional lacT. Alternatively, mutations in the transcriptional terminator can allow functional lacT transcription. Thus, inactivating mutations in the transcriptional terminator, in combination with a functional LacG, are sufficient to convert LGG to Lac+.

A preferred Lac+ bacterial strain is the L17 strain (ATCC Accession Number PTA-120536). L17 was derived from the L14 strain. The revertant mutation in the LacG stop codon resulted in increased LacG activity. Furthermore, L17 displayed good growth characteristics, i.e., larger colonies.

TABLE 1
Strains utilized over the course of this study

| | | | Lac |
|---|---|---|---|
| | | | pheno- |
| Strain | Genotype | Plasmid | type |
| L3 | Wild-type; | — | Lac− |
| (Lactobacillus rhamnosus | human isolate | | |
| ATCC 53103) | | | |
| L8 | Wild-type; | pTRKH3 | Lac− |
| | human isolate | | |
| L9 | Wild-type; | pTRKH3- | Lac− |
| | human isolate | erm-lacT | |
| | | (pG362) | |
| L11 | lacG (stop231K) | pG362 | Lac+ |
| L12 | lacG (stop232Q)) | pG362 | Lac+ |
| L13 | lacG (stop231K) | — | Lac− |
| L14 | lacG (stop231Q) | — | Lac− |
| L15 | lacG (stop231K); | — | Lac+ |
| | G to A | | |
| | mutation in | | |
| | transcriptional | | |
| | terminator | | |
| L16 | lacG (stop231K); | — | Lac+ |
| | G to T | | |
| | mutation in | | |
| | transcriptional | | |
| | terminator | | |
| L17 | lacG (stop231Q); | — | Lac+ |
| | G to A | | |
| | mutation in | | |
| | transcriptional | | |
| | terminator | | |
| L18 | lacG (stop231Q); | — | Lac+ |
| | G to T | | |
| | mutation in | | |
| | transcriptional | | |
| | terminator | | |

A plasmid transformation strategy was used in combination with successive rounds of plating to media containing lactose as the sole sugar source to isolate Lac+ LGG strains. These strains do not contain any foreign nucleic acids and are identical to the parental LGG strain except for 2 point mutations: (1) A point mutation in lacG that reverts a premature stop codon to a functional codon encoding glutamine or lysine and (2) a point mutation in the transcriptional terminator region within the lacTEGF promoter that removes the need for LacT and allows efficient transcription through the operon. As used herein, “foreign nucleic acids” refers to any DNA sequence that is not found in the Lactobacillus strain GG (ATCC 53103) (not including the mutations disclosed herein). Examples of foreign nucleic acids include, but are not limited to, exogenous plasmid-derived sequences, antibiotic resistance markers, introduced or incorporated into the LGG (ATCC 53103) chromosome during the process of converting the bacterium from Lac− to Lac+. These new Lac+ isolates of LGG retain all of the well-characterized probiotic benefits of the parental LGG strain, with the important new acquired advantage of efficient lactose utilization for growth. This Lac+ phenotype enables these strains to grow more robustly and more efficiently when administered in combination with foods that contain lactose (such as fermented milk/whey drinks, infant formula, buttermilk, cheeses, etc.). In this way, these strains more efficiently colonize and persist in the host, and therefore provide an enhanced probiotic benefit by growing to higher titers and thus exhibiting increased persistence. These Lac+ LGG isolates will also provide a means to treat lactose intolerance, again when provided in the context of a food containing lactose, such as yogurt. Finally, these Lac+ LGG strains offer utility as a starter culture for a wide range of fermented dairy products, a significant advantage over the Lac− parental LGG strain.

### Deposit of Biological Materials

Under the terms of the Budapest Treaty on the International Recognition of the Deposit of Microorganisms for the Purpose of Patent Procedure, the Lactobacillus rhamnosus GG Lac+ isolate L17 strain was deposited on Aug. 14, 2013, with the American Type Culture Collection (ATCC) of 10801 University Boulevard, Manassas, Va. 20110-2209 USA and was assigned ATCC Accession Number PTA-120536.

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

TABLE 2
Sequences

| SEQ ID | | |
|---|---|---|
| NO: | Description | Sequence |
| 1 | Nucleotide sequence of | ATGCCGAAAATCGATCAGATCTTTAACAATAACGTGG |
| | mutant lacT from strain | CGTTGGTTGAGCTAGACAACCATAGTCAAGCGGTTGTT |
| | Lactobacillus strain GG | AAGGGACGGGGGATCGCGTTTAGAAGCGTGGCGATGT |
| | (ATCC 53103) | GATTCCGGCTAAACAAATTGAAAAAATTTTCTACCTTG |
| | | CAAGTGAAACTTCCCGCCAGAATCTGTACTTTTTACTT |
| | | CGCAATATTCCGATCGACGTGGTGACGACCACTTATGA |
| | | AATTATTGACGTTGCGCAAAAACAATTTCACCTTAAGG |
| | | TACTTGACTATATTTACATTACGTTAAGCGATCATATT |
| | | TACGAAGCCTATAAACGTTATCAGAATGGAACGTATC |
| | | AAGAGACGATGGTGCCGGATTTTCATATTCAGTATCCT |
| | | GCAGAGTATGCCGTAGCTAACCAAGCGTTGCAGATCA |
| | | TTGCAGCTAACCTTGGCGTAGCATTTCCACCTTCCGAA |
| | | GTGAAAAATCTTGCCTTACATTTTATCAATGCCAGCGG |
| | | TGAAGACGACAACGAGCAGGCCTTTGACAAGAATAAT |
| | | GAGGCGTCATTAAGTCAACTGGTGCAACGTGTTTTGAA |
| | | GCGGCATCGCATTACGAGATCGCAAACTAACGGCAAT |
| | | TATTATGATCGCTTCATGATTCACTTACAGTATCTGATT |
| | | GACCGACTGCAGCGTGTTAATACGGATGCTGTTGCCAT |
| | | TGTGCCCGAGGTGGCCAATGAATTAGAGCTAAATTAT |
| | | CCGCGGTCTTATCAGATTGCTTCGGAAATTTTTGATGA |
| | | GATTAAGGATCAGCTTTATCGCAACATGAGTGAAGAC |
| | | GAACGCTTATACTTTATCATTCATATTCAACGGCTAAT |
| | | TAACGAAGCGCCTGCCCACGATCAAAAACAGGGCAAA |
| | | AAATCATAA |
| 2 | Amino acid sequence of | MPKIDQIFNNNVALVELDNHSQAVVKGRGIAFRSVAM* |
| | mutant LacT from strain | |
| | Lactobacillus strain GG | |
| | (ATCC 53103) (*= stop | |
| | codon) | |
| 3 | Nucleotide sequence of | ATGCGTAAACAATTACCCAAGGACTTTGTAATCGGTG |
| | mutant lacG from strain | GCGCAACTGCTGCTTACCAAGTTGAAGGGGCAACCAA |
| | Lactobacillus strain GG | AGAAGACGGAAAAGGTCGAGTTCTTTGGGATGATTTT |
| | (ATCC 53103) | CTGGAAAAACAAGGGCGGTTTAGTCCTGACCCCGCCG |
| | | CTGATTTTTATCATCGCTATGATGAGGATTTGGCGTTA |
| | | GCAGAAGCATATGGTCATCAAGTAATACGGCTTTCAA |
| | | TTGCCTGGTCGCGAATTTTTCCGGATGGTGCCGGGGCG |
| | | GTGGAACCTCGTGGCGTTGCTTTCTATCATCGGCTCTT |
| | | TGCTGCCTGTGCCAAGCATCATCTTATCCCGTTTGTAA |
| | | CGTTGCATCATTTTGATACACCAGAACGGTTACACGCG |
| | | ATTGGTGACTGGCTGAGTCAAGAAATGCTGGAAGATT |
| | | TTGTCGAGTACGCGCGGTTTTGCTTCGAGGAATTTCCG |
| | | GAAATCAAACACTGGATTACGATCAATGAACCAACGT |
| | | CCATGGCAGTGCAACAATATACGAGCGGCACTTTTCCC |
| | | CCAGCGGAAACCGGTCATTTTGATAAAACATTTCAGG |
| | | CCGAACATAATCAAATCGTTGCCCATGCGCGTATTGTT |
| | | AATTTGTACAAGTCGATGGGGCTAGATGGTGAAATCG |
| | | GTATCGTGCATGCCTTGCAGACACCTTATCCATATAGT |
| | | GATTCGTCGGAAGATTAGCATGCCGCTGATTTACAGG |
| | | ATGCGTTGGAAAATCGGCTGTATTTAGATGGCACACTG |
| | | GCAGGAGATTACGCCCCTAAGACCTTGGCTTTGATCAA |
| | | AGAAATTCTGGCAGCAAATCAACAACCGATGTTTAAG |
| | | CGTACCGACGAAGAGATGGCGGCTATTAAGAAGGCGG |
| | | CACACCAGCTTGATTTTGTTGGAGTTAACAACTACTTC |
| | | AGCAAATGGCTGCGCGCTTATCACGGCAAGTCGGAAA |
| | | CGATTCATAATGGTGATGGCTCAAAGGGATCGTCAGTT |
| | | GCCCGCCTTCACGGTATCGGTGAAGAGAAGAAACCGG |
| | | CCGGGATTGAGACAACGGATTGGGACTGGTCCATCTA |
| | | TCCGCGTGGTATGTATGACATGTTGATGCGGATTCATC |
| | | GAGATTATCCGTTAGTACCAGCCATCTATGTCACCGAA |
| | | AACGGTATTGGATTGAAAGAATCCTTACCAGCAGAAG |
| | | TGACGCCAAATACGGTCATCGCGGATCCCAAACGCAT |
| | | TGATTATTTGAAAAAATATTTAAGTGCCATTGCAGATG |
| | | CGATTCAGGCTGGCGCGAATGTAAAAGGCTACTTTGTC |
| | | TGGTCACTGCAGGATCAGTTTTCCTGGACAAATGGTTA |
| | | TAGCAAACGGTACGGATTGTTTTTCGTCGACTTTCCGA |
| | | CGCAAAAACGTTATGTCAAGCAAAGTGCCGAATGGTT |
| | | AAAACAAGTTAGCCAAACGCATGTGATTCCCGAATAA |
| 4 | Amino acid sequence of | MRKQLPKDFVIGGATAAYQVEGATKEDGKGRVLWDDF |
| | mutant LacG from strain | LEKQGRFSPDPAADFYHRYDEDLALAEAYGHQVIRLSIA |
| | Lactobacillus strain GG | WSRIFPDGAGAVEPRGVAFYHRLFAACAKHHLIPFVTLH |
| | (ATCC 53103) (*= stop | HFDTPERLHAIGDWLSQEMLEDFVEYARFCLLEFPEIKH |
| | codon) | WITINEPTSMAVQQYTSGTFPPAETGHFDKTFQAEHNQIV |
| | | AHARIVNLYKSMGLDGEIGIVHALQTPYPYSDSSED*HA |
| | | ADLQDALENRLYLDGTLAGDYAPKTLALIKEILAANQQP |
| | | MFKRTDEEMAAIKKAAHQLDFVGVNNYFSKWLRAYHG |
| | | KSETIHNGDGSKGSSVARLHGIGEEKKPAGIETTDWDWSI |
| | | YPRGMYDMLMRIHRDYPLVPAIYVTENGIGLKESLPAEV |
| | | TPNTVIADPKRIDYLKKYLSAIADAIQAGANVKGYFVWS |
| | | LQDQFSWTNGYSKRYGLEFVDFPTQKRYVKQSAEWLKQ |
| | | VSQTHVIP |
| 5 | Nucleotide sequence of | ATGCGTAAACAATTACCCAAGGACTTTGTAATCGGTG |
| | lacG from strain L11 | GCGCAACTGCTGCTTACCAAGTTGAAGGGGCAACCAA |
| | | AGAAGACGGAAAAGGTCGAGTTCTTTGGGATGATTTT |
| | | CTGGAAAAACAAGGGCGGTTTAGTCCTGACCCCGCCG |
| | | CTGATTTTTATCATCGCTATGATGAGGATTTGGCGTTA |
| | | GCAGAAGCATATGGTCATCAAGTAATACGGCTTTCAA |
| | | TTGCCTGGTCGCGAATTTTTCCGGATGGTGCCGGGGCG |
| | | GTGGAACCTCGTGGCGTTGCTTTCTATCATCGGCTCTT |
| | | TGCTGCCTGTGCCAAGCATCATCTTATCCCGTTTGTAA |
| | | CGTTGCATCATTTTGATACACCAGAACGGTTACACGCG |
| | | ATTGGTGACTGGCTGAGTCAAGAAATGCTGGAAGATT |
| | | TTGTCGAGTACGCGCGGTTTTGCTTCGAGGAATTTCCG |
| | | GAAATCAAACACTGGATTACGATCAATGAACCAACGT |
| | | CCATGGCAGTGCAACAATATACGAGCGGCACTTTTCCC |
| | | CCAGCGGAAACCGGTCATTTTGATAAAACATTTCAGG |
| | | CCGAACATAATCAAATCGTTGCCCATGCGCGTATTGTT |
| | | AATTTGTACAAGTCGATGGGGCTAGATGGTGAAATCG |
| | | GTATCGTGCATGCCTTGCAGACACCTTATCCATATAGT |
| | | GATTCGTCGGAAGATAAGCATGCCGCTGATTTACAGG |
| | | ATGCGTTGGAAAATCGGCTGTATTTAGATGGCACACTG |
| | | GCAGGAGATTACGCCCCTAAGACCTTGGCTTTGATCAA |
| | | AGAAATTCTGGCAGCAAATCAACAACCGATGTTTAAG |
| | | CGTACCGACGAAGAGATGGCGGCTATTAAGAAGGCGG |
| | | CACACCAGCTTGATTTTGTTGGAGTTAACAACTACTTC |
| | | AGCAAATGGCTGCGCGCTTATCACGGCAAGTCGGAAA |
| | | CGATTCATAATGGTGATGGCTCAAAGGGATCGTCAGTT |
| | | GCCCGCCTTCACGGTATCGGTGAAGAGAAGAAACCGG |
| | | CCGGGATTGAGACAACGGATTGGGACTGGTCCATCTA |
| | | TCCGCGTGGTATGTATGACATGTTGATGCGGATTCATC |
| | | GAGATTATCCGTTAGTACCAGCCATCTATGTCACCGAA |
| | | AACGGTATTGGATTGAAAGAATCCTTACCAGCAGAAG |
| | | TGACGCCAAATACGGTCATCGCGGATCCCAAACGCAT |
| | | TGATTATTTGAAAAAATATTTAAGTGCCATTGCAGATG |
| | | CGATTCAGGCTGGCGCGAATGTAAAAGGCTACTTTGTC |
| | | TGGTCACTGCAGGATCAGTTTTCCTGGACAAATGGTTA |
| | | TAGCAAACGGTACGGATTGTTTTTCGTCGACTTTCCGA |
| | | CGCAAAAACGTTATGTCAAGCAAAGTGCCGAATGGTT |
| | | AAAACAAGTTAGCCAAACGCATGTGATTCCCGAATAA |
| 6 | Amino acid sequence of | MRKQLPKDFVIGGATAAYQVEGATKEDGKGRVLWDDF |
| | LacG from strain L11 | LEKQGRFSPDPAADFYHRYDEDLALAEAYGHQVIRLSIA |
| | (*= stop codon) | WSRIFPDGAGAVEPRGVAFYHRLFAACAKHHLIPFVTLH |
| | | HFDTPERLHAIGDWLSQEMLEDFVEYARFCFEEFPEIKH |
| | | WITINEPTSMAVQQYTSGTFPPAETGHFDKTFQAEHNQIV |
| | | AHARIVNLYKSMGLDGEIGIVHALQTPYPYSDSSEDKHA |
| | | ADLQDALENRLYLDGTLAGDYAPKTLALIKEILAANQQP |
| | | MFKRTDEEMAAIKKAAHQLDFVGVNNYFSKWLRAYHG |
| | | KSETIHNGDGSKGSSVARLHGIGEEKKPAGIETTDWDWSI |
| | | YPRGMYDMLMRIHRDYPLVPAIYVTENGIGLKESLPAEV |
| | | TPNTVIADPKRIDYLKKYLSAIADAIQAGANVKGYFVWS |
| | | LQDQFSWTNGYSKRYGLFFVDFPTQKRYVKQSAEWLKQ |
| | | VSQTHVIPE* |
| 7 | Nucleotide sequence of | ATGCGTAAACAATTACCCAAGGACTTTGTAATCGGTG |
| | lacG from strain L12 | GCGCAACTGCTGCTTACCAAGTTGAAGGGGCAACCAA |
| | | AGAAGACGGAAAAGGTCGAGTTCTTTGGGATGATTTT |
| | | CTGGAAAAACAAGGGCGGTTTAGTCCTGACCCCGCCG |
| | | CTGATTTTTATCATCGCTATGATGAGGATTTGGCGTTA |
| | | GCAGAAGCATATGGTCATCAAGTAATACGGCTTTCAA |
| | | TTGCCTGGTCGCGAATTTTTCCGGATGGTGCCGGGGCG |
| | | GTGGAACCTCGTGGCGTTGCTTTCTATCATCGGCTCTT |
| | | TGCTGCCTGTGCCAAGCATCATCTTATCCCGTTTGTAA |
| | | CGTTGCATCATTTTGATACACCAGAACGGTTACACGCG |
| | | ATTGGTGACTGGCTGAGTCAAGAAATGCTGGAAGATT |
| | | TTGTCGAGTACGCGCGGTTTTGCTTCGAGGAATTTCCG |
| | | GAAATCAAACACTGGATTACGATCAATGAACCAACGT |
| | | CCATGGCAGTGCAACAATATACGAGCGGCACTTTTCCC |
| | | CCAGCGGAAACCGGTCATTTTGATAAAACATTTCAGG |
| | | CCGAACATAATCAAATCGTTGCCCATGCGCGTATTGTT |
| | | AATTTGTACAAGTCGATGGGGCTAGATGGTGAAATCG |
| | | GTATCGTGCATGCCTTGCAGACACCTTATCCATATAGT |
| | | GATTCGTCGGAAGATCAGCATGCCGCTGATTTACAGG |
| | | ATGCGTTGGAAAATCGGCTGTATTTAGATGGCACACTG |
| | | GCAGGAGATTACGCCCCTAAGACCTTGGCTTTGATCAA |
| | | AGAAATTCTGGCAGCAAATCAACAACCGATGTTTAAG |
| | | CGTACCGACGAAGAGATGGCGGCTATTAAGAAGGCGG |
| | | CACACCAGCTTGATTTTGTTGGAGTTAACAACTACTTC |
| | | AGCAAATGGCTGCGCGCTTATCACGGCAAGTCGGAAA |
| | | CGATTCATAATGGTGATGGCTCAAAGGGATCGTCAGTT |
| | | GCCCGCCTTCACGGTATCGGTGAAGAGAAGAAACCGG |
| | | CCGGGATTGAGACAACGGATTGGGACTGGTCCATCTA |
| | | TCCGCGTGGTATGTATGACATGTTGATGCGGATTCATC |
| | | GAGATTATCCGTTAGTACCAGCCATCTATGTCACCGAA |
| | | AACGGTATTGGATTGAAAGAATCCTTACCAGCAGAAG |
| | | TGACGCCAAATACGGTCATCGCGGATCCCAAACGCAT |
| | | TGATTATTTGAAAAAATATTTAAGTGCCATTGCAGATG |
| | | CGATTCAGGCTGGCGCGAATGTAAAAGGCTACTTTGTC |
| | | TGGTCACTGCAGGATCAGTTTTCCTGGACAAATGGTTA |
| | | TAGCAAACGGTACGGATTGTTTTTCGTCGACTTTCCGA |
| | | CGCAAAAACGTTATGTCAAGCAAAGTGCCGAATGGTT |
| | | AAAACAAGTTAGCCAAACGCATGTGATTCCCGAATAA |
| 8 | Amino acid sequence of | MRKQLPKDFVIGGATAAYQVEGATKEDGKGRVLWDDF |
| | LacG from strain L12 | LEKQGRFSPDPAADFYHRYDEDLALAEAYGHQVIRLSIA |
| | (*= stop codon) | WSRIFPDGAGAVEPRGVAFYHRLFAACAKHHLIPFVTLH |
| | | HFDTPERLHAIGDWLSQEMLEDFVEYARFCFEEFPEIKH |
| | | WITINEPTSMAVQQYTSGTFPPAETGHFDKTFQAEHNQIV |
| | | AHARIVNLYKSMGLDGEIGIVHALQTPYPYSDSSEDQHA |
| | | ADLQDALENRLYLDGTLAGDYAPKTLALIKEILAANQQP |
| | | MFKRTDEEMAAIKKAAHQLDFVGVNNYFSKWLRAYHG |
| | | KSETIHNGDGSKGSSVARLHGIGEEKKPAGIETTDWDWSI |
| | | YPRGMYDMLMRIHRDYPLVPAIYVTENGIGLKESLPAEV |
| | | TPNTVIADPKRIDYLKKYLSAIADAIQAGANVKGYFVWS |
| | | LQDQFSWTNGYSKRYGLFFVDFPTQKRYVKQSAEWLKQ |
| | | VSQTHVIPE* |
| 9 | pG362 Nucleotide | GGATCCACAGGACGGGTGTGGTCGCCATGATCGCGTA |
| | Sequence | GTCGATAGTGGCTCCAAGTAGCGAAGCGAGCAGGACT |
| | | GGGCGGCGGCCAAAGCGGTCGGACAGTGCTCCGAGAA |
| | | CGGGTGCGCATAGAAATTGCATCAACGCATATAGCGC |
| | | TAGCAGCACGCCATAGTGACTGGCGATGCTGTCGGAA |
| | | TGGACGATATCCCGCAAGAGGCCCGGCAGTACCGGCA |
| | | TAACCAAGCCTATGCCTACAGCATCCAGGGTGACGGT |
| | | GCCGAGGATGACGATGAGCGCATTGTTAGATTTCATA |
| | | CACGGTGCCTGACTGCGTTAGCAATTTAACTGTGATAA |
| | | ACTACCGCATTAAAGCTTATCGATGATAAGCTGTCAAA |
| | | CATGAGAATTACAACTTATATCGTATGGGGCTGACTTC |
| | | AGGTGCTACATTTGAAGAGATAAATTGCACTGAAATC |
| | | TAGAAATATTTTATCTGATTAATAAGATGATCTTCTTG |
| | | AGATCGTTTTGGTCTGCGCGTAATCTCTTGCTCTGAAA |
| | | ACGAAAAAACCGCCTTGCAGGGCGGTTTTTCGAAGGT |
| | | TCTCTGAGCTACCAACTCTTTGAACCGAGGTAACTGGC |
| | | TTGGAGGAGCGCAGTCACCAAAACTTGTCCTTTCAGTT |
| | | TAGCCTTAACCGGCGCATGACTTCAAGACTAACTCCTC |
| | | TAAATCAATTACCAGTGGCTGCTGCCAGTGGTGCTTTT |
| | | GCATGTCTTTCCGGGTTGGACTCAAGACGATAGTTACC |
| | | GGATAAGGCGCAGCGGTCGGACTGAACGGGGGGTTCG |
| | | TGCATACAGTCCAGCTTGGAGCGAACTGCCTACCCGG |
| | | AACTGAGTGTCAGGCGTGGAATGAGACAAACGCGGCC |
| | | ATAACAGCGGAATGACACCGGTAAACCGAAAGGCAG |
| | | GAACAGGAGAGCGCACGAGGGAGCCGCCAGGGGGAA |
| | | ACGCCTGGTATCTTTATAGTCCTGTCGGGTTTCGCCAC |
| | | CACTGATTTGAGCGTCAGATTTCGTGATGCTTGTCAGG |
| | | GGGGCGGAGCCTATGGAAAAACGGCTTTGCCGCGGCC |
| | | CTCTCACTTCCCTGTTAAGTATCTTCCTGGCATCTTCCA |
| | | GGAAATCTCCGCCCCGTTCGTAAGCCATTTCCGCTCGC |
| | | CGCAGTCGAACGACCGAGCGTAGCGAGTCAGTGAGCG |
| | | AGGAAGCGGAATATATCCTGTATCACATATTCTGCTGA |
| | | CGCACCGGTGCAGCCTTTTTTCTCCTGCCACATGAAGC |
| | | ACTTCACTGACACCCTCATCAGTGCCAACATAGTAAGC |
| | | CAGTATACACTCCGCTAGCGCTGATGTCCGGCGGTGCT |
| | | TTTGCCGTTACGCACCACCCCGTCAGTAGCTGAACAGG |
| | | AGGGACAGCTGATAGAAACAGAAGCCACTGGAGCACC |
| | | TCAAAAACACCATCATACACTAAATCAGTAAGTTGGC |
| | | AGCATCACCCGACGCACTTTGCGCCGAATAAATACCT |
| | | GTGACGGAAGATCACTTCGCAGAATAAATAAATCCTG |
| | | GTGTCCCTGTTGATACCGGGAAGCCCTGGGCCAACTTT |
| | | TGGCGAAAATGAGACGTTGATCGGCACGTAAGAGGTT |
| | | CCAACTTTCACCATAATGAAATAAGATCACTACCGGG |
| | | CGTATTTTTTGAGTTATCGAGATTTTCAGGAGCTAAGG |
| | | AAGCTAAAATGGAGAAAAAAATCACTGGATATACCAC |
| | | CGTTGATATATCCCAATGGCATCGTAAAGAACATTTTG |
| | | AGGCATTTCAGTCAGTTGCTCAATGTACCTATAACCAG |
| | | ACCGTTCAGCTGGATATTACGGCCTTTTTAAAGACCGT |
| | | AAAGAAAAATAAGCACAAGTTTTATCCGGCCTTTATTC |
| | | ACATTCTTGCCCGCCTGATGAATGCTCATCCGGAATTC |
| | | TATTTAATCACTTTGACTAGCAAATACTAACAACAAGA |
| | | CACACACACCAAAAATCAAAAATTCACTACTTTTAGTT |
| | | AAAAACCACGTAACCACAAGAACTAATCCAATCCATG |
| | | TAATCGGGTTCTTCAAATATTTCTCCAAGATTTTCCTCC |
| | | TCTAATATGCTCAACTTAAATGACCTATTCAATAAATC |
| | | TATTATGCTGCTAAATAGTTTATAGGACAAATAAGTAT |
| | | ACTCTAATGACCTATAAAAGATAGAAAATTAAAAAAT |
| | | CAAGTGTTCGCTTCGCTCTCACTGCCCCTCGACGTTTT |
| | | AGTAGCCTTTCCCTCACTTCGTTCAGTCCAAGCCAACT |
| | | AAAAGTTTTCGGGCTACTCTCTCCTTCTCCCCCTAATA |
| | | ATTAATTAAAATCTTACTCTGTATATTTCTGCTAATCAT |
| | | TCGCTAAACAGCAAAGAAAAAACAAACACGTATCATA |
| | | GATATAAATGTAATGGCATAGTGCGGGTTTTATTTTCA |
| | | GCCTGTATCATAGCTAAACAAATCGAGTTGTGTGTCCG |
| | | TTTTAGGGCGTTCTGCTAGCTTGTTTAAAGTCTCTTGA |
| | | ATGAATGTATGCTCTAAGTCAAAAGAATTTGTCAGCGC |
| | | CTTTATATAGCTTTCTTTTTCTTCTTTTTTTACTTTAATG |
| | | ATCGATAGCAACAATGATTTAACACTAGCAAGTTGAA |
| | | TGCCACCATTTCTTCCTGGTTTAATCTTAAAGAAAATT |
| | | TCCTGATTCGCCTTCAGTACCTTCAGCAATTTATCTAAT |
| | | GTCCGTTCAGGAATGCCTAGCACTTCTCTAATCTCTTTT |
| | | TTGGTCGTCACTAAATAAGGCTTGTATACATCGCTTTT |
| | | TTCGCTAATATAAGCCATTAAATCTTCTTTCCATTCTGA |
| | | CAAATGAACACGTTGACGTTCGCTTCTTTTTTTCTTGA |
| | | ATTTAAACCACCCTTGACGGACAAATAAATCTTTACTG |
| | | GTTAAATCACTTGATACCCAAGCTTTGCAAAGAATGGT |
| | | AATGTATTCCCTATTAGCCCCTTGATAGTTTTCTGAAT |
| | | AGGCACTTCTAACAATTTTGATTACTTCTTTTTCTTCTA |
| | | AGGGTTGATCTAATCGATTATTAAACTCAAACATATTA |
| | | TATTCGCACGTTTCGATTGAATAGCCTGAACTAAAGTA |
| | | GGCTAAAGAGAGGGTAAACATGACGTTATTACGCCCT |
| | | ATTAAACCCTTTTCTCCTGAAAATTTCGTTTCGTGCAAT |
| | | AAGAGATTAAACCAGGGTTCATCTACTTGTTTTTTGCC |
| | | TTCTGTACCGCTTAAAACCGTTAGACTTGAACGAGTAA |
| | | AGCCCTTATTATCTGTTTGTTTGAAAGACCAATCTTGC |
| | | CATTCTTTGAAAGAATAACGGTAATTAGGATCAAAAA |
| | | ATTCTACATTGTCCGTTCTTGGTATGCGAGCAATACCA |
| | | AAATGATTACACGTTAGATCAACTGGCAAAGACTTTCC |
| | | AAAATATTCTCGGATATTTTGCGAAATTATTTTGGCTG |
| | | CTTTGACAGATTTAAATTCTGATTTTGAAGTCACATAG |
| | | ACTGGCGTTTCTAAAACAAAATATGCTTGATAACCTTT |
| | | ATCAGATTTGATAATCATAGTAGGCATAAAACCTAAA |
| | | TCAATAGCGGTTGTTAAAATATCGCTTGCTGAAATAGT |
| | | TTCTTTTGCCGTGTGAATATCAAAATCAATAAAGAAGG |
| | | TATTGATTTGTCTTAAATTGTTTTCAGAATGTCCTTTCG |
| | | TGTATGAACGGTTTTCGTCTGCATACGTTCCATAACGA |
| | | TAAACGTTGGGTGTCCAATGTGTAAATGTATCTTGATT |
| | | TTCTTGAATCGCTTCCTCGGAAGTCAGAACAACACCAC |
| | | GACCGCCAATCATGCTTGATTTTGAGCGATACGCAAA |
| | | AATAGCCCCTTTGCTTTTACCTGGCTTGGTAGTGATTG |
| | | AGCGAATTTTACTATTTTTAAATTTGTACTTTAACAAG |
| | | CCGTCATGAAGCACAGTTTCTACAACAAAAGGGATAT |
| | | TCATTCAGCTGTTCTCCTTTCCTATAAATCCTATAAAAT |
| | | AGGTTGTTTAATTAACTTGGTTTGCTTTTTCATTCAACT |
| | | GTTTCAATATTGCATGTTTTGAAAAAGATTTTTTTCCTT |
| | | TATAAGTCAATTTTTTTCCACTAATCGAATAAATTATTT |
| | | TGTTATTTTCTATTAACTTATATATATAATCTTCCCCCT |
| | | CCGAAGAAAAATACTTATCTGATTTTGTTTCTAAGTAG |
| | | ATATTTCTCTTTTCTAACTCTTTCTTAAACGTTTCTAGT |
| | | GTATAGATATTTGCTAATTTTCTTATCTCCAATAAACT |
| | | ATTTTTTATATAAGTTTTACATTCATCATGATTCATACA |
| | | AACTCCACCTTCTATAAATGAATACAAAAAAAGCAAT |
| | | CAAACGATTTCCGATTGATTGCTTAACAATTCTTAAAT |
| | | TCAGTAGCTTAGATACTTGAAAACTCTCTGATTTCCCT |
| | | ATATAATGATAGTACGGTTATATACCGTCTTCAAACAA |
| | | AGTTAATTAAATAACTTCTTACGAGGGAAGAGTTCATC |
| | | TGACTAACTGATAAGCGTTGGTTTGGCAATCTTATCGG |
| | | GCTATGCATTTATAAAATGTCGTCAAACATTTTATAAA |
| | | TGTGTCATGGCTCTTTTTTCGTTTCTATTCAGTTCGTTG |
| | | TTTCGTTATATCTAGTATACCGCTTTTAAAAAAAAATA |
| | | AGCAACGATTTCGTGCATTATTCACACGAAGTCATTGC |
| | | TTTTTTCTTCTTCCATTTCTAAATCCAATGTTACTTGTT |
| | | CTGATTCTGTTTCTGGCTCTGGTTCTGTTGGCTCATTTG |
| | | GGATTAAATCCACTACTAGCGTTGAGTTAGTTAACTTT |
| | | GCAATTTGTTCTAGTGTTTTTATGGTTGGATCTGATTTT |
| | | CCTGATTCTATTCGTGAATAATTTGATCTACTCATTTCT |
| | | AATTCTTGGGGTACCGCCAGCATTTCGGAAAAAAACC |
| | | ACGCTAAGGATTTTTTCTATAAAAAGAGCCGTTATATT |
| | | AAGAATAAAACGGCTCTTTTATACGTAAAGGACGTAA |
| | | ATTCATTTGCCCAGTGTCATGTAATCCTTCAAATTTGT |
| | | ATTCTCCAAGAAAATTGATATGTTCCCATCCTAACGGC |
| | | CACGCATATGGCATTAAATCTTCTCTAAATTCTCCTCTT |
| | | GCTTTTAATTCTTCTACGGCTTTTTCCATATATACAGTG |
| | | TTCCACACACTTATAGCGTTAATAATTATGTTTAGTGC |
| | | ACTAGCTCTTTGTAACTGGTCTTGGAGAGCACGTTCTC |
| | | TAAATTCTCCACGTTGTCCAAAAAATATAGTTCTAGCT |
| | | AATGCATTGATTGCTTCTCCTTTATTTAAACCTTTTTGA |
| | | ACCCGTCTCCTTACGGCTTTATTAGATATGTAATCCAG |
| | | CGTAAAGAGGGTTTTCTCGATTCGTCCCATTTCTCCAA |
| | | GTGCTGTTGCGAGTTTATTTTGTCTTGCATATGATCCG |
| | | AGCTTCCCCATGATAAGAGCGCTAGGGACCTCTTTAGC |
| | | TCCTTGGAAGCTGTCAGTAGTATACCTAATAATTTATC |
| | | TACATTCCCTTTAGTAACGTGTAACTTTCCAAATTTAC |
| | | AAAAGCGACTCATAGAATTATTTCCTCCCGTTAAATAA |
| | | TAGATAACTATTAAAAATAGACAATACTTGCTCATAA |
| | | GTAACGGTACTTAAATTGTTTACTTTGGCGTGTTTCATT |
| | | GCTTGATGAAACTGATTTTTAGTAAACAGTTGACGATA |
| | | TTCTCGATTGACCCATTTTGAAACAAAGTACGTATATA |
| | | GCTTCCAATATTTATCTGGAACATCTGTGGTATGGCGG |
| | | GTAAGTTTTATTAAGACACTGTTTACTTTTGGTTTAGG |
| | | ATGAAAGCATTCCGCTGGCAGCTTAAGCAATTGCTGA |
| | | ATCGAGACTTGAGTGTGCAAGAGCAACCCTAGTGTTC |
| | | GGTGAATATCCAAGGTACGCTTGTAGAATCCTTCTTCA |
| | | ACAATCAGATAGATGTCAGACGCATGGCTTTCAAAAA |
| | | CCACTTTTTTAATAATTTGTGTGCTTAAATGGTAAGGA |
| | | ATACTCCCAACAATTTTATACCTCTGTTTGTTAGGGAA |
| | | TTGAAACTGTAGAATATCTTGGTGAATTAAAGTGACAC |
| | | GAGTATTCAGTTTTAATTTTTCTGACGATAAGTTGAAT |
| | | AGATGACTGTCTAATTCAATAGACGTTACCTGTTTACT |
| | | TATTTTAGCCAGTTTCGTCGTTAAATGCCCTTTACCTGT |
| | | TCCAATTTCGTAAACGGTATCGGTTTCTTTTAAATTCA |
| | | ATTGTTTTATTATTTGGTTGAGTACTTTTTCACTCGTTA |
| | | AAAAGTTTTGAGAATATTTTATATTTTTGTTCATGTAAT |
| | | CACTCCTTCTTAATTACAAATTTTTAGCATCTAATTTAA |
| | | CTTCAATTCCTATTATACAAAATTTTAAGATACTGCAC |
| | | TATCAACACACTCTTAAGTTTGCTTCTAAGTCTTATTTC |
| | | CATAACTTCTTTTACGTTTCCGCCATTCTTTGCTGTTTC |
| | | GATTTTTATGATATGGTGCAAGTCAGCACGAACACGA |
| | | ACCGTCTTATCTCCCATTATATCTTTTTTTGCACTGATT |
| | | GGTGTATCATTTCGTTTTTCTTTTTGTGCGCTTCTTGAT |
| | | AAAAGGGATAGTAATTCATTCCTGGTTGCAAATTTTGA |
| | | AAACCGCTACGGATCACATCTTTTTCTAAACTATTGAT |
| | | CCATAGTCTTTTATACGTTTTATCTTTAGAAAAGGCAT |
| | | TTGCTTTATGAATGATCGACCAGGCAATGTTTTCGCCT |
| | | TCTCTGTCGCTATCTGTTGCGACAATGATTGTATTTGCT |
| | | TGTTTTAAAAGTTCAGCAACAATTTTAAACTGCTTTTTT |
| | | TTATCTGTTGCCACTTCAAAATCGTATCGATTCTAGAC |
| | | TCGAGATGCGCCGCGTGCGGCTGCTGGAGATGGCGGA |
| | | CGCGATGGATATGTTCTGCCAAGGGTTGGTTTGCGCAT |
| | | TCACAGTTCTCCGCAAGAATTGATTGGCTCCAATTCTT |
| | | GGAGTGGTGAATCCGTTAGCGAGGTGCCGCCGGCTTC |
| | | CATTCAGGTCGAGGTGGCCCGGCTCCATGCACCGCGA |
| | | CGCAACGCGGGGAGGCAGACAAGGTATAGGGCGGCG |
| | | CCTACAATCCATGCCAACCCGTTCCATGTGCTCGCCGA |
| | | GGCGGCATAAATCGCCGTGACGATCAGCGGTCCAGTG |
| | | ATCGAAGTTAGGCTGGTAAGAGCCGCGAGCGATCCTT |
| | | GAAGCTGTCCCTGATGGTCGTCATCTACCTGCCTGGAC |
| | | AGCATGGCCTGCAACGCGGGCATCCCGATGCCGCCGG |
| | | AAGCGAGAAGAATCATAATGGGGAAGGCCATCCAGCC |
| | | TCGCGTCGCGAACGCCAGCAAGACGTAGCCCAGCGCG |
| | | TCGGCCGCCATGCCGGCGATAATGGCCTGCTTCTCGCC |
| | | GAAACGTTTGGTGGCGGGACCAGTGACGAAGGCTTGA |
| | | GCGAGGGCGTGCAAGATTCCGAATACCGCAAGCGACA |
| | | GGCCGATCATCGTCGCGCTCCAGCGAAAGCGGTCCTC |
| | | GCCGAAAATGACCCAGAGCGCTGCCGGCACCTGTCCT |
| | | ACGAGTTGCATGATAAAGAAGACAGTCATAAGTGCGG |
| | | CGACGATAGTCATGCCCCGCGCCCACCGGAAGGAGCT |
| | | GACTGGGTTGAAGGCTCTCAAGGGCATCGGTCGACAG |
| | | TCTAGAATCGATACGATTTTGAAGTGGCAACAGATAA |
| | | AAAAAAGCAGTTTAAAATTGTTGCTGAACTTTTAAAAC |
| | | AAGCAAATACAATCATTGTCGCAACAGATAGCGACAG |
| | | AGAAGGCGAAAACATTGCCTGGTCGATCATTCATAAA |
| | | GCAAATGCCTTTTCTAAAGATAAAACGTATAAAAGAC |
| | | TATGGATCAATAGTTTAGAAAAAGATGTGATCCGTAG |
| | | CGGTTTTCAAAATTTGCAACCAGGAATGAATTACTATC |
| | | CCTTTTATCAAGAAGCGCACAAAAAGAAAAACGAAAT |
| | | GATACACCAATCAGTGCAAAAAAAGATATAATGGGAG |
| | | ATAAGACGGTTCGTGTTCGTGCTGACTTGCACCATATC |
| | | ATAAAAATCGAAACAGCAAAGAATGGCGGAAACGTA |
| | | AAAGAAGTTATGGAAATAAGACTTAGAAGCAAACTTA |
| | | AGAGTGTGTTGATAGTGCAGTATCTTAAAATTTTGTAT |
| | | AATAGGAATTGAAGTTAAATTAGATGCTAAAAATTTG |
| | | TAATTAGCGGCCGCAGAAGGAGTGAATTCATGCCGAA |
| | | AATCGATCAGATCTTTAACAATAACGTGGCGTTGGTTG |
| | | AGCTAGACAACCATAGTCAAGCGGTTGTTAAGGGACG |
| | | GGGGATCGCGTTTCAAAAGAAGCGTGGCGATGTGATT |
| | | CCGGCTAAACAAATTGAAAAAATTTTCTACCTTGCAAG |
| | | TGAAACTTCCCGCCAGAATCTGTACTTTTTACTTCGCA |
| | | ATATTCCGATCGACGTGGTGACGACCACTTATGAAATT |
| | | ATTGACGTTGCGCAAAAACAATTTCACCTTAAGGTACT |
| | | TGACTATATTTACATTACGTTAAGCGATCATATTTACG |
| | | AAGCCTATAAACGTTATCAGAATGGAACGTATCAAGA |
| | | GACGATGGTGCCGGATTTTCATATTCAGTATCCTGCAG |
| | | AGTATGCCGTAGCTAACCAAGCGTTGCAGATCATTGC |
| | | AGCTAACCTTGGCGTAGCATTTCCACCTTCCGAAGTGA |
| | | AAAATCTTGCCTTACATTTTATCAATGCCAGCGGTGAA |
| | | GACGACAACGAGCAGGCCTTTGACAAGAATAATGAGG |
| | | CGTCATTAAGTCAACTGGTGCAACGTGTTTTGAAGCGG |
| | | CATCGCATTACGAGATCGCAAACTAACGGCAATTATT |
| | | ATGATCGCTTCATGATTCACTTACAGTATCTGATTGAC |
| | | CGACTGCAGCGTGTTAATACGGATGCTGTTGCCATTGT |
| | | GCCCGAGGTGGCCAATGAATTAGAGCTAAATTATCCG |
| | | CGGTCTTATCAGATTGCTTCGGAAATTTTTGATGAGAT |
| | | TAAGGATCAGCTTTATCGCAACATGAGTGAAGACGAA |
| | | CGCTTATACTTTATCATTCATATTCAACGGCTAATTAA |
| | | CGAAGCGCCTGCCCACGATCAAAAACAGGGCAAAAAA |
| | | TCATAAACTAGT |
| 10 | Nucleic acid sequence of | ATGTTGCTAAAGGTCAAGCAAGTCTATAACAATAATG |
| | lacT from L. rhamnosus | CTGTACTTGTAGACGTTGGCAACGGTAAAGAGGCGAT |
| | Lc705 | TCTTCAGGGGAAAGGAATTGGTTTCAGCAAGCGTAAA |
| | | GGTGATGACGTTGATCCAAAAGCGAGTTCGGAAATAC |
| | | TTTATCTAAATGATACGCAAGTCAAAAATCACTTTACA |
| | | TCTTTACTCAAAGATGTACCGATTGACATTGTTGTTGC |
| | | TACATTTGGTGTGATCGGAATGGCAAAAAAGAAGTAT |
| | | CACTATCCGGTTTTGAACTATATTTATGTCACACTTAC |
| | | GGACCACGTCTTTCAGATGTATAAACGATTGACTGCCG |
| | | GCAAATATCAAGCTAGTCCAGCGCCTGACATCCGTGA |
| | | TCGTTACCCGTTGCCTTACCAGATCGCAGCCGATGCGC |
| | | GCCGACAGCTTAACCATGATCTTGGCGTTCAGTTTCCT |
| | | GAAGCCGAAATTAAGAATATTGCCTTACATTTTATTAA |
| | | TGCAAAGGGAGTGGATGGCGAACTGGATCCGACCGTG |
| | | ACCCTGACGGCGCGCGTCAATGCAATCGTGACACAAG |
| | | TATTTGCTAAATATGGGTTAAATCGCAATTTTGCTAAT |
| | | CAAAATTACTTCGATCGGCTAATGATTCATTTGCAATA |
| | | CCTTGTTGAACGTTTAAATACCAATGAGCAAGACGAG |
| | | GCTGACCTTGGTCCGGAAATTGGTCAGGACTTCAGGC |
| | | GCCTTTATCCCAAATCGTTTACGATCGCAACTGAAATT |
| | | TGTACGGAGCTGGAAAAAGCCTTACAAATTAAGCTCA |
| | | ACGAAAACGAGCATGTGTACTTTATTATCCATATTCAA |
| | | CGGCTCATCCAAGAACCGCAAACACTTCCGCCTGAAT |
| | | ATCCATAG |
| 11 | Amino acid sequence of | MLLKVKQVYNNNAVLVDVGNGKEAILQGKGIGFSKRKG |
| | LacT from L. rhamnosus | DDVDPKASSEILYLNDTQVKNHFTSLLKDVPIDIVVATFG |
| | Lc705 | VIGMAKKKYHYPVLNYIYVTLTDHVFQMYKRLTAGKY |
| | | QASPAPDIRDRYPLPYQIAADARRQLNHDLGVQFPEAEIK |
| | | NIALHFINAKGVDGELDPTVTLTARVNAIVTQVFAKYGL |
| | | NRNFANQNYFDRLMIHLQYLVERLNTNEQDEADLGPEIG |
| | | QDFRRLYPKSFTIATEICTELEKALQIKLNENEHVYFIIHIQ |
| | | RLIQEPQTLPPEYP* |
| 12 | L15 | CCACAAATCATGACGCTGAGAGTAGCGCGTGATTTGTT |
| 13 | L16 | CCACAAATCATGACGCTGAGAGTAGCGCGTGATTTGTA |
| 14 | L17 | CCACAAATCATGACGCTGAGAGTAGCGCGTGATTTGTA |
| 15 | L18 | CCACAAATCATGACGCTGAGAGTAGCGCGTGATTTGTT |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A Lactobacillus rhamnosus bacterial strain comprising at least 99% identity to the nucleotide sequence of strain ATCC 53103 and further comprising a gene encoding a functional LacG protein and a gene encoding a functional LacT protein or a lacTEGF operon lacking a functional transcriptional terminator within 100 nucleotide base pairs from the initiating codon of lacT.

2. The bacterial strain of claim 1 comprising at least 99.9% identity to the nucleotide sequence of strain ATCC 53103.

3. The bacterial strain of claim 1, wherein the gene encoding a functional LacG protein comprises the amino acid sequence SEQ ID NO: 6 or 8.

4. The bacterial strain of claim 3, wherein the gene encoding a functional LacG protein comprises the nucleotide sequence SEQ ID NO: 5 or 7.

5. The bacterial strain of claim 1, wherein the gene encoding a functional LacT protein comprises the amino acid sequence SEQ ID NO: 11.

6. The bacterial strain of claim 5, wherein the gene encoding a functional LacT protein comprises the nucleotide sequence SEQ ID NO: 10.

7. The bacterial strain of claim 1, wherein the lacTEGF operon lacking a functional transcriptional terminator within 100 nucleotide base pairs from the initiating codon of lacT comprises any one of the nucleotide sequences SEQ ID NO: 12-15.

8. A Lactobacillus rhamnosus bacterial strain comprising at least 99% of the nucleotide sequence of strain ATCC 53103 and further comprising one or more of the following:
a. at least one mutation that suppresses the LacT defect found in the strain ATCC 53103;
b. at least one mutation in the transcriptional terminator regulating the lacTEGF operon, wherein the at least one mutation inactivates the transcriptional terminator;
c. the nucleotide sequence encoding a functional LacT or the amino acid SEQ ID NO: 11; and
d. the nucleotide sequence encoding a functional LacG or the amino acid sequence of SEQ ID NOS: 6 or 8.

9. The bacterial strain of claim 8 comprising at least 99.9% of the nucleotide sequence of strain ATCC 53103.

10. The bacterial strain of claim 8, wherein the mutation in the transcriptional terminator comprises the nucleotide sequence of SEQ ID NOs: 12-15.

11. The bacterial strain of claim 8, wherein the nucleotide sequence encoding functional LacT comprises SEQ ID NO: 10.

12. The bacterial strain of claim 8, wherein the nucleotide sequence encoding functional LacG comprises SEQ ID NOs: 5 or 7.

13. An ingestible composition comprising lactose and the strain of claim 1.

14. The composition of claim 13, wherein said compositions comprises infant formula, milk drink, whey protein drink, sport drink, juices, buttermilk, cheese, yogurt, drinkable yogurt, baby food, weaning food, or confections such as candy or gum.

15. A yogurt starter culture comprising the strain of claim 1.

16. A method of making yogurt comprising contacting a dairy product with any of the bacteria of claim 1.

17. A bacterial culture comprising any of the bacteria of claim 1.

18. The bacterial culture of claim 7, further comprising any one of the following bacterial strains:
Acetobacter orientalis, Bifidobacterium animalis, Bifidobacterium lactis, Bifidobacterium bifidum, Lactobacillus acidophilus, Lactobacillus bulgaricus, Lactobacillus casei, Lactobacillus delbrueckii subsp. Bulgaricus, Lactobacillus delbrueckii subsp. Lactis, Lactobacillus lactis subsp. Cremoris, Lactobacillus rhamnosus, Lactococcus lactis, Leuconostoc mesenteroides, Streptococcus lactis var. bollandicus, Streptococcus taette, and Streptococcus thermophiles.

19. A method of identifying a bacteria that can digest lactose comprising:
a. transforming a Lactobacillus rhamnosus with a first plasmid comprising functional or wild-type LacT and a selective marker;
b. selecting for at least one Lac+ colony by growing the transformants of step (a) on plates containing lactose and the selective marker,
c. passaging the Lac+ colony of step (b) at least two times in the absence of lactose and the selective marker, such that the Lac+ colonies no longer contain the first plasmid;
d. growing the Lac+ colony of step (c) on plates containing lactose, thereby identifying a bacteria that can digest lactose.

20. The Lactobacillus rhamnosus bacterial strain of claim 1, wherein said strain comprises ATCC Accession Number PTA-120536.


SEQUENCE LISTING (15 sequences)

SEQ ID NO: 1 - DNA, 875 bp, Artificial Sequence
  altered sequence
atgccgaaaatcgatcagatctttaacaataacgtggcgttggttgagctagacaaccatagtcaagcggttgttaagggacgggggatcgcgtttagaagcgtggcgatgtgattccggctaaacaaattgaaaaaattttctaccttgcaagtgaaacttcccgccagaatctgtactttttacttcgcaatattccgatcgacgtggtgacgaccacttatgaaattattgacgttgcgcaaaaacaatttcaccttaaggtacttgactatatttacattacgttaagcgatcatatttacgaagcctataaacgttatcagaatggaacgtatcaagagacgatggtgccggattttcatattcagtatcctgcagagtatgccgtagctaaccaagcgttgcagatcattgcagctaaccttggcgtagcatttccaccttccgaagtgaaaaatcttgccttacattttatcaatgccagcggtgaagacgacaacgagcaggcctttgacaagaataatgaggcgtcattaagtcaactggtgcaacgtgttttgaagcggcatcgcattacgagatcgcaaactaacggcaattattatgatcgcttcatgattcacttacagtatctgattgaccgactgcagcgtgttaatacggatgctgttgccattgtgcccgaggtggccaatgaattagagctaaattatccgcggtcttatcagattgcttcggaaatttttgatgagattaaggatcagctttatcgcaacatgagtgaagacgaacgcttatactttatcattcatattcaacggctaattaacgaagcgcctgcccacgatcaaaaacagggcaaaaaatcataa

SEQ ID NO: 2 - protein, 37 aa, Artificial Sequence
  altered sequence
MPKIDQIFNNNVALVELDNHSQAVVKGRGIAFRSVAM

SEQ ID NO: 3 - DNA, 1425 bp, Artificial Sequence
  altered sequence
atgcgtaaacaattacccaaggactttgtaatcggtggcgcaactgctgcttaccaagttgaaggggcaaccaaagaagacggaaaaggtcgagttctttgggatgattttctggaaaaacaagggcggtttagtcctgaccccgccgctgatttttatcatcgctatgatgaggatttggcgttagcagaagcatatggtcatcaagtaatacggctttcaattgcctggtcgcgaatttttccggatggtgccggggcggtggaacctcgtggcgttgctttctatcatcggctctttgctgcctgtgccaagcatcatcttatcccgtttgtaacgttgcatcattttgatacaccagaacggttacacgcgattggtgactggctgagtcaagaaatgctggaagattttgtcgagtacgcgcggttttgcttcgaggaatttccggaaatcaaacactggattacgatcaatgaaccaacgtccatggcagtgcaacaatatacgagcggcacttttcccccagcggaaaccggtcattttgataaaacatttcaggccgaacataatcaaatcgttgcccatgcgcgtattgttaatttgtacaagtcgatggggctagatggtgaaatcggtatcgtgcatgccttgcagacaccttatccatatagtgattcgtcggaagattagcatgccgctgatttacaggatgcgttggaaaatcggctgtatttagatggcacactggcaggagattacgcccctaagaccttggctttgatcaaagaaattctggcagcaaatcaacaaccgatgtttaagcgtaccgacgaagagatggcggctattaagaaggcggcacaccagcttgattttgttggagttaacaactacttcagcaaatggctgcgcgcttatcacggcaagtcggaaacgattcataatggtgatggctcaaagggatcgtcagttgcccgccttcacggtatcggtgaagagaagaaaccggccgggattgagacaacggattgggactggtccatctatccgcgtggtatgtatgacatgttgatgcggattcatcgagattatccgttagtaccagccatctatgtcaccgaaaacggtattggattgaaagaatccttaccagcagaagtgacgccaaatacggtcatcgcggatcccaaacgcattgattatttgaaaaaatatttaagtgccattgcagatgcgattcaggctggcgcgaatgtaaaaggctactttgtctggtcactgcaggatcagttttcctggacaaatggttatagcaaacggtacggattgtttttcgtcgactttccgacgcaaaaacgttatgtcaagcaaagtgccgaatggttaaaacaagttagccaaacgcatgtgattcccgaataa

SEQ ID NO: 4 - protein, 472 aa, Artificial Sequence
  altered sequence
MRKQLPKDFVIGGATAAYQVEGATKEDGKGRVLWDDFLEKQGRFSPDPAADFYHRYDEDLALAEAYGHQVIRLSIAWSRIFPDGAGAVEPRGVAFYHRLFAACAKHHLIPFVTLHHFDTPERLHAIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDHAADLQDALENRLYLDGTLAGDYAPKTLALIKEILAANQQPMFKRTDEEMAAIKKAAHQLDFVGVNNYFSKWLRAYHGKSETIHNGDGSKGSSVARLHGIGEEKKPAGIETTDWDWSIYPRGMYDMLMRIHRDYPLVPAIYVTENGIGLKESLPAEVTPNTVIADPKRIDYLKKYLSAIADAIQAGANVKGYFVWSLQDQFSWTNGYSKRYGLFFVDFPTQKRYVKQSAEWLKQVSQTHVIP

SEQ ID NO: 5 - DNA, 1425 bp, Artificial Sequence
  altered sequence
atgcgtaaacaattacccaaggactttgtaatcggtggcgcaactgctgcttaccaagttgaaggggcaaccaaagaagacggaaaaggtcgagttctttgggatgattttctggaaaaacaagggcggtttagtcctgaccccgccgctgatttttatcatcgctatgatgaggatttggcgttagcagaagcatatggtcatcaagtaatacggctttcaattgcctggtcgcgaatttttccggatggtgccggggcggtggaacctcgtggcgttgctttctatcatcggctctttgctgcctgtgccaagcatcatcttatcccgtttgtaacgttgcatcattttgatacaccagaacggttacacgcgattggtgactggctgagtcaagaaatgctggaagattttgtcgagtacgcgcggttttgcttcgaggaatttccggaaatcaaacactggattacgatcaatgaaccaacgtccatggcagtgcaacaatatacgagcggcacttttcccccagcggaaaccggtcattttgataaaacatttcaggccgaacataatcaaatcgttgcccatgcgcgtattgttaatttgtacaagtcgatggggctagatggtgaaatcggtatcgtgcatgccttgcagacaccttatccatatagtgattcgtcggaagataagcatgccgctgatttacaggatgcgttggaaaatcggctgtatttagatggcacactggcaggagattacgcccctaagaccttggctttgatcaaagaaattctggcagcaaatcaacaaccgatgtttaagcgtaccgacgaagagatggcggctattaagaaggcggcacaccagcttgattttgttggagttaacaactacttcagcaaatggctgcgcgcttatcacggcaagtcggaaacgattcataatggtgatggctcaaagggatcgtcagttgcccgccttcacggtatcggtgaagagaagaaaccggccgggattgagacaacggattgggactggtccatctatccgcgtggtatgtatgacatgttgatgcggattcatcgagattatccgttagtaccagccatctatgtcaccgaaaacggtattggattgaaagaatccttaccagcagaagtgacgccaaatacggtcatcgcggatcccaaacgcattgattatttgaaaaaatatttaagtgccattgcagatgcgattcaggctggcgcgaatgtaaaaggctactttgtctggtcactgcaggatcagttttcctggacaaatggttatagcaaacggtacggattgtttttcgtcgactttccgacgcaaaaacgttatgtcaagcaaagtgccgaatggttaaaacaagttagccaaacgcatgtgattcccgaataa

SEQ ID NO: 6 - protein, 474 aa, Artificial Sequence
  altered sequence
MRKQLPKDFVIGGATAAYQVEGATKEDGKGRVLWDDFLEKQGRFSPDPAADFYHRYDEDLALAEAYGHQVIRLSIAWSRIFPDGAGAVEPRGVAFYHRLFAACAKHHLIPFVTLHHFDTPERLHAIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDKHAADLQDALENRLYLDGTLAGDYAPKTLALIKEILAANQQPMFKRTDEEMAAIKKAAHQLDFVGVNNYFSKWLRAYHGKSETIHNGDGSKGSSVARLHGIGEEKKPAGIETTDWDWSIYPRGMYDMLMRIHRDYPLVPAIYVTENGIGLKESLPAEVTPNTVIADPKRIDYLKKYLSAIADAIQAGANVKGYFVWSLQDQFSWTNGYSKRYGLFFVDFPTQKRYVKQSAEWLKQVSQTHVIPE

SEQ ID NO: 7 - DNA, 1425 bp, Artificial Sequence
  altered sequence
atgcgtaaacaattacccaaggactttgtaatcggtggcgcaactgctgcttaccaagttgaaggggcaaccaaagaagacggaaaaggtcgagttctttgggatgattttctggaaaaacaagggcggtttagtcctgaccccgccgctgatttttatcatcgctatgatgaggatttggcgttagcagaagcatatggtcatcaagtaatacggctttcaattgcctggtcgcgaatttttccggatggtgccggggcggtggaacctcgtggcgttgctttctatcatcggctctttgctgcctgtgccaagcatcatcttatcccgtttgtaacgttgcatcattttgatacaccagaacggttacacgcgattggtgactggctgagtcaagaaatgctggaagattttgtcgagtacgcgcggttttgcttcgaggaatttccggaaatcaaacactggattacgatcaatgaaccaacgtccatggcagtgcaacaatatacgagcggcacttttcccccagcggaaaccggtcattttgataaaacatttcaggccgaacataatcaaatcgttgcccatgcgcgtattgttaatttgtacaagtcgatggggctagatggtgaaatcggtatcgtgcatgccttgcagacaccttatccatatagtgattcgtcggaagatcagcatgccgctgatttacaggatgcgttggaaaatcggctgtatttagatggcacactggcaggagattacgcccctaagaccttggctttgatcaaagaaattctggcagcaaatcaacaaccgatgtttaagcgtaccgacgaagagatggcggctattaagaaggcggcacaccagcttgattttgttggagttaacaactacttcagcaaatggctgcgcgcttatcacggcaagtcggaaacgattcataatggtgatggctcaaagggatcgtcagttgcccgccttcacggtatcggtgaagagaagaaaccggccgggattgagacaacggattgggactggtccatctatccgcgtggtatgtatgacatgttgatgcggattcatcgagattatccgttagtaccagccatctatgtcaccgaaaacggtattggattgaaagaatccttaccagcagaagtgacgccaaatacggtcatcgcggatcccaaacgcattgattatttgaaaaaatatttaagtgccattgcagatgcgattcaggctggcgcgaatgtaaaaggctactttgtctggtcactgcaggatcagttttcctggacaaatggttatagcaaacggtacggattgtttttcgtcgactttccgacgcaaaaacgttatgtcaagcaaagtgccgaatggttaaaacaagttagccaaacgcatgtgattcccgaataa

SEQ ID NO: 8 - protein, 474 aa, Artificial Sequence
  altered sequence
MRKQLPKDFVIGGATAAYQVEGATKEDGKGRVLWDDFLEKQGRFSPDPAADFYHRYDEDLALAEAYGHQVIRLSIAWSRIFPDGAGAVEPRGVAFYHRLFAACAKHHLIPFVTLHHFDTPERLHAIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDQHAADLQDALENRLYLDGTLAGDYAPKTLALIKEILAANQQPMFKRTDEEMAAIKKAAHQLDFVGVNNYFSKWLRAYHGKSETIHNGDGSKGSSVARLHGIGEEKKPAGIETTDWDWSIYPRGMYDMLMRIHRDYPLVPAIYVTENGIGLKESLPAEVTPNTVIADPKRIDYLKKYLSAIADAIQAGANVKGYFVWSLQDQFSWTNGYSKRYGLFFVDFPTQKRYVKQSAEWLKQVSQTHVIPE

SEQ ID NO: 9 - DNA, 8936 bp, Artificial Sequence
  altered sequence
ggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattctatttaatcactttgactagcaaatactaacaacaagacacacacaccaaaaatcaaaaattcactacttttagttaaaaaccacgtaaccacaagaactaatccaatccatgtaatcgggttcttcaaatatttctccaagattttcctcctctaatatgctcaacttaaatgacctattcaataaatctattatgctgctaaatagtttataggacaaataagtatactctaatgacctataaaagatagaaaattaaaaaatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcgctaaacagcaaagaaaaaacaaacacgtatcatagatataaatgtaatggcatagtgcgggttttattttcagcctgtatcatagctaaacaaatcgagttgtgtgtccgttttagggcgttctgctagcttgtttaaagtctcttgaatgaatgtatgctctaagtcaaaagaatttgtcagcgcctttatatagctttctttttcttctttttttactttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcactaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacatgacgttattacgccctattaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattaggatcaaaaaattctacattgtccgttcttggtatgcgagcaataccaaaatgattacacgttagatcaactggcaaagactttccaaaatattctcggatattttgcgaaattattttggctgctttgacagatttaaattctgattttgaagtcacatagactggcgtttctaaaacaaaatatgcttgataacctttatcagatttgataatcatagtaggcataaaacctaaatcaatagcggttgttaaaatatcgcttgctgaaatagtttcttttgccgtgtgaatatcaaaatcaataaagaaggtattgatttgtcttaaattgttttcagaatgtcctttcgtgtatgaacggttttcgtctgcatacgttccataacgataaacgttgggtgtccaatgtgtaaatgtatcttgattttcttgaatcgcttcctcggaagtcagaacaacaccacgaccgccaatcatgcttgattttgagcgatacgcaaaaatagcccctttgcttttacctggcttggtagtgattgagcgaattttactatttttaaatttgtactttaacaagccgtcatgaagcacagtttctacaacaaaagggatattcattcagctgttctcctttcctataaatcctataaaataggttgtttaattaacttggtttgctttttcattcaactgtttcaatattgcatgttttgaaaaagatttttttcctttataagtcaatttttttccactaatcgaataaattattttgttattttctattaacttatatatataatcttccccctccgaagaaaaatacttatctgattttgtttctaagtagatatttctcttttctaactctttcttaaacgtttctagtgtatagatatttgctaattttcttatctccaataaactattttttatataagttttacattcatcatgattcatacaaactccaccttctataaatgaatacaaaaaaagcaatcaaacgatttccgattgattgcttaacaattcttaaattcagtagcttagatacttgaaaactctctgatttccctatataatgatagtacggttatataccgtcttcaaacaaagttaattaaataacttcttacgagggaagagttcatctgactaactgataagcgttggtttggcaatcttatcgggctatgcatttataaaatgtcgtcaaacattttataaatgtgtcatggctcttttttcgtttctattcagttcgttgtttcgttatatctagtataccgcttttaaaaaaaaataagcaacgatttcgtgcattattcacacgaagtcattgcttttttcttcttccatttctaaatccaatgttacttgttctgattctgtttctggctctggttctgttggctcatttgggattaaatccactactagcgttgagttagttaactttgcaatttgttctagtgtttttatggttggatctgattttcctgattctattcgtgaataatttgatctactcatttctaattcttggggtaccgccagcatttcggaaaaaaaccacgctaaggattttttctataaaaagagccgttatattaagaataaaacggctcttttatacgtaaaggacgtaaattcatttgcccagtgtcatgtaatccttcaaatttgtattctccaagaaaattgatatgttcccatcctaacggccacgcatatggcattaaatcttctctaaattctcctcttgcttttaattcttctacggctttttccatatatacagtgttccacacacttatagcgttaataattatgtttagtgcactagctctttgtaactggtcttggagagcacgttctctaaattctccacgttgtccaaaaaatatagttctagctaatgcattgattgcttctcctttatttaaacctttttgaacccgtctccttacggctttattagatatgtaatccagcgtaaagagggttttctcgattcgtcccatttctccaagtgctgttgcgagtttattttgtcttgcatatgatccgagcttccccatgataagagcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgccattctttgctgtttcgatttttatgatatggtgcaagtcagcacgaacacgaaccgtcttatctcccattatatctttttttgcactgattggtgtatcatttcgtttttctttttgtgcgcttcttgataaaagggatagtaattcattcctggttgcaaattttgaaaaccgctacggatcacatctttttctaaactattgatccatagtcttttatacgttttatctttagaaaaggcatttgctttatgaatgatcgaccaggcaatgttttcgccttctctgtcgctatctgttgcgacaatgattgtatttgcttgttttaaaagttcagcaacaattttaaactgcttttttttatctgttgccacttcaaaatcgtatcgattctagactcgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacagtctagaatcgatacgattttgaagtggcaacagataaaaaaaagcagtttaaaattgttgctgaacttttaaaacaagcaaatacaatcattgtcgcaacagatagcgacagagaaggcgaaaacattgcctggtcgatcattcataaagcaaatgccttttctaaagataaaacgtataaaagactatggatcaatagtttagaaaaagatgtgatccgtagcggttttcaaaatttgcaaccaggaatgaattactatcccttttatcaagaagcgcacaaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattagcggccgcagaaggagtgaattcatgccgaaaatcgatcagatctttaacaataacgtggcgttggttgagctagacaaccatagtcaagcggttgttaagggacgggggatcgcgtttcaaaagaagcgtggcgatgtgattccggctaaacaaattgaaaaaattttctaccttgcaagtgaaacttcccgccagaatctgtactttttacttcgcaatattccgatcgacgtggtgacgaccacttatgaaattattgacgttgcgcaaaaacaatttcaccttaaggtacttgactatatttacattacgttaagcgatcatatttacgaagcctataaacgttatcagaatggaacgtatcaagagacgatggtgccggattttcatattcagtatcctgcagagtatgccgtagctaaccaagcgttgcagatcattgcagctaaccttggcgtagcatttccaccttccgaagtgaaaaatcttgccttacattttatcaatgccagcggtgaagacgacaacgagcaggcctttgacaagaataatgaggcgtcattaagtcaactggtgcaacgtgttttgaagcggcatcgcattacgagatcgcaaactaacggcaattattatgatcgcttcatgattcacttacagtatctgattgaccgactgcagcgtgttaatacggatgctgttgccattgtgcccgaggtggccaatgaattagagctaaattatccgcggtcttatcagattgcttcggaaatttttgatgagattaaggatcagctttatcgcaacatgagtgaagacgaacgcttatactttatcattcatattcaacggctaattaacgaagcgcctgcccacgatcaaaaacagggcaaaaaatcataaactagt

SEQ ID NO: 10 - DNA, 870 bp, Artificial Sequence
  altered sequence
atgttgctaaaggtcaagcaagtctataacaataatgctgtacttgtagacgttggcaacggtaaagaggcgattcttcaggggaaaggaattggtttcagcaagcgtaaaggtgatgacgttgatccaaaagcgagttcggaaatactttatctaaatgatacgcaagtcaaaaatcactttacatctttactcaaagatgtaccgattgacattgttgttgctacatttggtgtgatcggaatggcaaaaaagaagtatcactatccggttttgaactatatttatgtcacacttacggaccacgtctttcagatgtataaacgattgactgccggcaaatatcaagctagtccagcgcctgacatccgtgatcgttacccgttgccttaccagatcgcagccgatgcgcgccgacagcttaaccatgatcttggcgttcagtttcctgaagccgaaattaagaatattgccttacattttattaatgcaaagggagtggatggcgaactggatccgaccgtgaccctgacggcgcgcgtcaatgcaatcgtgacacaagtatttgctaaatatgggttaaatcgcaattttgctaatcaaaattacttcgatcggctaatgattcatttgcaataccttgttgaacgtttaaataccaatgagcaagacgaggctgaccttggtccggaaattggtcaggacttcaggcgcctttatcccaaatcgtttacgatcgcaactgaaatttgtacggagctggaaaaagccttacaaattaagctcaacgaaaacgagcatgtgtactttattatccatattcaacggctcatccaagaaccgcaaacacttccgcctgaatatccatag

SEQ ID NO: 11 - protein, 289 aa, Artificial Sequence
  altered sequence
MLLKVKQVYNNNAVLVDVGNGKEAILQGKGIGFSKRKGDDVDPKASSEILYLNDTQVKNHFTSLLKDVPIDIVVATFGVIGMAKKKYHYPVLNYIYVTLTDHVFQMYKRLTAGKYQASPAPDIRDRYPLPYQIAADARRQLNHDLGVQFPEAEIKNIALHFINAKGVDGELDPTVTLTARVNAIVTQVFAKYGLNRNFANQNYFDRLMIHLQYLVERLNTNEQDEADLGPEIGQDFRRLYPKSFTIATEICTELEKALQIKLNENEHVYFIIHIQRLIQEPQTLPPEYP

SEQ ID NO: 12 - DNA, 38 bp, Artificial Sequence
  altered sequence
ccacaaatcatgacgctgagagtagcgcgtgatttgtt

SEQ ID NO: 13 - DNA, 38 bp, Artificial Sequence; identical to SEQ ID NO: 12
  altered sequence

SEQ ID NO: 14 - DNA, 38 bp, Artificial Sequence
  altered sequence
ccacaaatcatgacgctgagagtagcgcgtgatttgta

SEQ ID NO: 15 - DNA, 38 bp, Artificial Sequence; identical to SEQ ID NO: 12
  altered sequence